(19) United States
(12) Patent Application Publication
(10) Pub. No.: US 2021/0196933 A1
(43) Pub. Date: Jul. 1, 2021

(54) KIT FOR PREVENTING DISTAL EMBOLISM DURING ENDOVASCULAR INTERVENTIONS AND METHOD FOR PREVENTING DISTAL EMBOLISM DURING ENDOVASCULAR INTERVENTIONS USING SAID KIT

(71) Applicants: Zaza KAVTELADZE, Potomac, MD (US); David KAVTELADZE, Potomac, MD (US)

(72) Inventors: Zaza KAVTELADZE, Potomac, MD (US); David KAVTELADZE, Potomac, MD (US)

(21) Appl. No.: 17/115,072

(22) Filed: Dec. 8, 2020

(30) Foreign Application Priority Data
Dec. 30, 2019 (RU) 2019145158

(51) Int. Cl. A61M 25/10

(52) U.S. Cl. CPC A61M 25/1011; A61M 2025/1061; A61M 2025/1079; A61M 2210/125; A61M 2205/7545; A61M 2205/0216; A61M 2210/127; A61M 2025/109

(57) ABSTRACT

Devices and methods are disclosed for preventing distal embolisms during endovascular interventions. Representative devices are kits that include a first catheter comprising a proximal part and distal part having a principal through lumen, at least one lumen for filling at least one balloon, at least one inflated balloon that closes the lumen of the right or left carotid artery, and a proximal end configured to allow blood to flow through the principal lumen but to block the passage of emboli. The kits include a second catheter in the form of a single-lumen tube with a proximal end and a distal end. The proximal and distal parts of the first catheter may have diameters that are sufficient to allow passage through the lumen of the second catheter and may be sufficiently long when combined to allow at least the distal part of the first catheter to extend beyond the limits of the distal end of the second catheter, when the first catheter passes through the second catheter. Moreover, the internal diameter of the lumen of the second catheter may exceed the external diameter of the distal part of the first catheter.

### CROSS-REFERENCE TO RELATED APPLICATIONS

This application claims priority to Russian patent application RU 2019145158, filed Dec. 30, 2019 (published as RU 2725552C1 on Jul. 2, 2020). Benefit of the filing date of this application under 35 U.S.C. § 119 is hereby claimed. This application is hereby incorporated by reference in its entirety.

### FIELD OF THE INVENTION

This group of inventions relates to the field of endovascular surgery, and specifically to a kit for and methods of preventing distal embolism during endovascular interventions, including the prevention of strokes.

### DESCRIPTION OF RELATED ART

An intra-aortic embolism is one of the most frightening complications of cardiac, aortic and vascular procedures, and is diagnosed in 1-22% of patients who undergo cardiovascular operations. Even more frequently, in 70% of cases, patients who undergo insertion of shunts into the coronary arteries, heart valve replacements and aortic surgery suffer sub-clinical embolisms, which can be registered using transcranial Doppler graphs and MRT. These embolism phenomena can cause cognitive problems and disabilities and have a significant effect on patients' recoveries.

The principal sources of cerebral (distal) embolisms and strokes in this situation are the heart, the heart valves, the thoracic aorta and the major vessels when surgery is performed on them. Even simple catheterisation of the heart using an endovascular catheter may cause micro-trauma to an atherosclerotic thoracic trauma, leading to formation of embolus particles and subsequent embolic damage to the brain caused by latent ischaemic foci and leading to massive and even fatal strokes.

The prior art refers to many devices which have attempted to prevent carotid artery embolisms during endovascular and cardiac operations. These anti-embolus devices, however, have not been widely used in vascular, heart valve or thoracic aorta surgery because of their complexity and invasive nature with a risk of additional trauma to the inner walls of vessels, leading to a high risk-benefit ratio. The prior art requires additional equipment to be introduced into the arterial system or aorta, a procedure which in itself, as is known, is connected with all the classic risks of endovascular intervention, including aortic dissection, haemorrhage, thrombosis, carotid artery embolism and stroke.

One prior art is an intra-aortic filtering device, which is introduced into the ascending thoracic aorta through an aortic cannula to catch potential embolus material released from the heart and the aortic walls during heart surgery. However, this device is relatively difficult to produce, and its use has been linked with serious injury to the aortic wall and acute aortic dissection. Another device for preventing embolisms in cerebral blood circulation includes a porous deflector/ordinary aortic screen, which catches or takes away a potential embolism to the distal vascular network. Still another device was proposed for use during aortic valve surgery and takes the form of an intra-aortic filtering catheter which catches emboli during the procedure in question. Embolus formation can also be caused by poor positioning of the filter on the aortic wall or carotid artery wall.

Therefore, despite the numerous innovations in the field of anti-embolus devices, the problem of distal cerebral embolism and stroke during cardiovascular surgery is far from being solved.

The use of occlusive balloon catheters to prevent embolism is also known. These occlusive balloon catheters are positioned inside one of the carotid arteries when endovascular interventions are carried out on the heart and aorta.

The most similar device (prototype) to the group of inventions declared is a stroke prevention device (US 2017/0165457 A1, 15, Jun. 2017), which contains a first occlusive catheter, which in turn contains a distal part and a proximal part and has a principal lumen for changing the pressure, one lumen for filling one balloon and one lumen designed to be fixed in the right common carotid artery; a second occlusive catheter containing a distal part and a proximal part and having a principal lumen for measuring pressure, one lumen for filling one balloon and one balloon designed to be fixed in the left common carotid artery; and a device for introducing the catheters, produced in the form of a three-lumen tube with a proximal end and a distal end; in this case the proximal and distal parts have external diameters that are such as to allow passage of the device to be introduced through the lumens, this meaning that the internal diameters of the lumens on the device for introducing the catheters are greater than the external diameter of the first and second occlusive catheters.

The principal shortcoming of this prototype is the complete blockage of blood flow during endovascular operations on heart valves, in at least two arteries that supply the brain. The duration of the blockage is variable; it depends on the complexity and duration of the surgical procedure, which may cause ischaemic brain damage, especially in elderly patients, who are the group for whom endovascular heart valve implants are most frequently indicated.

The technical problem that needs to be resolved when producing and using the group of inventions declared, one which could not be solved when producing and using the similar inventions, is that of maintaining cerebral blood flow while preventing distal emboli from entering the arteries in the head, which would allow any operation of any complexity, unlimited in time where necessary, to be carried out without the fear of ischaemic brain damage and with full and effective protection against distal emboli.

### SUMMARY OF THE INVENTION

The technical result provided by the declared group of inventions in terms of resolution of the technical problem mentioned above is the existence of a complete blockage on distal embolisms entering the arteries in the head while maintaining blood flow in the distal section of these arteries.

The above-mentioned problem is resolved, and the technical result achieved, by using the proposed devices for preventing distal embolisms during endovascular interventions and the methods for preventing distal embolism during endovascular interventions using said devices.

Moreover, in accordance with a first embodiment, the kit for preventing distal embolisms during endovascular interventions includes the following: a first catheter comprising a proximal part and distal part having a principal through lumen, at least one lumen for filling at least one balloon, at least one inflated balloon which closes the lumen of the right or left carotid artery, and a proximal end produced so as to allow blood to flow through the above-mentioned principal lumen but to block the passage of emboli through it; and a second catheter produced in the form of a single-lumen tube with a proximal end and a distal end; moreover, the proximal and distal parts of the first catheter have diameters that are sufficient to allow passage through the lumen of the second catheter and are sufficiently long when combined to allow at least the distal part of the first catheter to extend beyond the limits of the distal end of the second catheter, when the first catheter passes through the second catheter; moreover, the internal diameter of the lumen of the second catheter exceeds the external diameter of the distal part of the first catheter.

In addition, the proximal part of the first catheter consists of at least one lumen for filling at least one balloon, while the distal part of the first catheter takes the form of a tube with at least two lumens, which includes: a principal through lumen the internal diameter of which allows blood to flow through the above-mentioned principal lumen, and at least one lumen for filling at least one balloon, which is narrower than the principal through lumen and is connected with the above-mentioned at least one lumen for filling at least one balloon in the proximal part of the first catheter.

In addition, at least one lumen for filling at least one balloon of the distal part of the first catheter ends at the point of positioning of at least one balloon, while the principal through lumen passes through the place of positioning of at least one balloon and to the distal end of the distal part of the first catheter.

In addition, at least one balloon is made from an elastomer material.

Additionally, the distal part of the first catheter is made from radio-opaque material or contains at least one radio-opaque marker.

In addition, the distal part of the first catheter has a principal through lumen, one lumen for filling one balloon, and one inflated balloon which covers the lumen of the right or left common carotid artery and is positioned from the side of the distal end on said distal part, while the proximal part of the first catheter consists of one lumen for filling one balloon.

Also, the proximal end of the principal through lumen of the distal part of the first catheter is an anti-embolus filter.

In this case, in keeping with one of the aspects, the anti-embolus filter consists of a set of through holes made in the wall of the principal through lumen at the proximal end of the principal through filter of the distal part of the first catheter.

The principal through lumen of the distal part of the first catheter is cylindrical throughout its length.

Here, the above-mentioned holes made in the wall of the principal through lumen are positioned not less than 20 cm and not more than 70 cm from the balloon and measure 50 to 300 microns (μm), and more preferably to 100 μm, in diameter.

In addition, the principal through lumen of the distal part of the first catheter narrows conically at its proximal end.

In this case, the length of the conically narrowing proximal end of the principal through lumen of the distal part of the first catheter is within a range of 2 to 40 cm, preferably to 30 cm and more preferably to 20 cm.

The above-mentioned holes made in the wall of the principal through lumen are 50 to 300 μm and more preferably to 100 μm in diameter.

In keeping with the additional aspect, the anti-embolus filter takes the form of a braided, knitted or meshed conical polymer filter.

In this case, the length of the conical filter is within a range from 2 cm to 40 cm, preferably to 30 cm and more preferably to 20 cm.

In keeping with another aspect, the anti-embolus filter takes the form of a network that closes the proximal hole in the principal through lumen.

According to one more aspect, the anti-embolus filter takes the form of a porous membrane that closes the proximal opening of the principal through lumen.

In addition, the distal part of the first catheter has a principal through lumen, two lumens for filling two balloons and two balloons positioned at a distance from each other and inflated; this inflation covers the lumen of the right or left common carotid artery, while the proximal part of the first catheter consists of two lumens for filling two balloons.

Also, the proximal end of the principal through lumen of the distal part of the first catheter is an anti-embolus filter.

In this case, the anti-embolus filter takes the form of a braided, knitted or meshed polymer conical filter.

In addition, the anti-embolus filter takes the form of a network that covers the proximal hole in the principal through lumen.

The anti-embolus filter also takes the form of a porous membrane which covers the proximal hole in the principal through lumen.

In addition, the distance between the balloons is 3-4 cm.

Also, at the end-point of the proximal end of the principal through lumen of the distal part of the first catheter is a hole equal in diameter to that of the guidewire that passes through said hole into the principal through lumen of the distal part of the first catheter.

In addition, the principal through lumen of the distal part of the first catheter has an internal diameter of not less than 1 mm, but not more than 2.5 mm.

In addition, the distal part of the first catheter has an external diameter of not less than 1.2 mm, but not more than 2.8 mm.

In addition, the proximal end of at least one lumen for filling at least one balloon in the proximal part of the first catheter is closed by an elastic material that allows an injection needle to pass through.

Also, the distal end of the principal through lumen of the distal part of the first catheter is closed with an additional anti-embolus filter.

In addition, the kit contains a third catheter, made in the form of a single-lumen tine with a proximal end and a distal end; in this case, the proximal and distal parts of the first catheter both have internal diameters sufficient to allow a third catheter to pass through the lumen and have lengths that together are sufficient for at least the distal part of the first catheter to extend beyond the limits of the distal end of the third catheter, when the first catheter passes through the third catheter; in this case, the internal diameter of the lumen of the third catheter is in essence equal in size to the external diameter of the distal part of the first catheter.

In accordance with a second embodiment, the kit for preventing distal embolism during endovascular interventions includes: a first catheter comprising a proximal part and a distal part, which has a principal through lumen, which in turn is cylindrical throughout its length, a lumen for filling the balloon, an inflated balloon which closes the lumen of the right or left common carotid artery and is positioned on the above-mentioned distal part from its distal end side, and an anti-embolus filter placed on the proximal end of the principal through lumen of the distal part of the first catheter; and a second catheter made in the form of a single-lumen tube with a proximal end and a distal end; in this case, the proximal and distal parts of the first catheter have internal diameters sufficient to pass through the lumen of the second catheter and also have a combined length sufficient for at least the distal part of the first catheter to extend beyond the limits of the distal end of the second catheter when the first catheter passes through the second catheter; in this case the internal diameter of the lumen of the second catheter is greater than that of the external diameter of the distal part of the first catheter; in this case the anti-embolus filter comprises a series of through holes made in the wall of the principal through lumen on the distal part of the first catheter and positioned not less than 20 cm and not more than 70 cm from the balloon and measuring not less than 50 cm and not more than 300 μm in diameter.

In addition, the proximal part of the first catheter consists of one lumen for filling the balloon, while the distal part of the first catheter takes the form of a two-lumen tube, comprising a principal through lumen the internal diameter of which is sufficient to allow blood to flow through said principal lumen, and the lumen for filling the balloon, which is narrower than the principal through lumen and is connected to the previously mentioned lumen for fulling the balloon in the proximal part of the first catheter.

In addition, the lumen for filling balloons in the distal part of the first catheter ends at the place of positioning of the balloon, while the principal through lumen passes through the place of positioning of the balloon and as far as the distal end of the distal part of the first catheter.

In addition, the balloon is made of an elastomer material.

Also, the distal part of the first catheter is made of radio-opaque material or contains at least one radio-opaque marker.

Additionally, the principal through lumen of the distal part of the first catheter has an internal diameter of not less than 1 mm, but not more than 2.5 mm.

Additionally, the distal part of the first catheter has an external diameter of not less than 1.2 mm, but not more than 2.8 mm.

In addition, the proximal end of the lumen for filling the balloon in the proximal part of the first catheter is closed with an elastic material that allows an injection needle to pass through.

In addition, the distal end of the principal through lumen of the distal part of the first catheter is closed with an additional anti-embolus filter.

Additionally, the kit contains a third catheter, made in the form of a single-lumen tube, with a proximal end and a distal end; in this case, the proximal and distal parts of the first catheter have external diameters sufficient to allow passage through the lumen of the third catheter and combined length sufficient to allow at least the distal part of the first catheter to extend beyond the limits of the distal end of the third catheter, when the first catheter passes through the third catheter; in this case, the internal diameter of the lumen of the third catheter is equal to the external diameter of the distal part of the first catheter.

In accordance with a third embodiment, the kit for preventing distal embolism during endovascular interventions includes the following: a first catheter comprising a proximal part and distal part with a principal through lumen that narrows conically at its proximal end, a lumen for filling the balloon, an inflated balloon that closes the lumen of the right or left common carotid artery and is positioned on the above-mentioned distal part from the side of its distal end, and an anti-embolus filter placed on the proximal end of the principal through lumen of the distal part of the first catheter; and a second catheter made in the form of a single-lumen tube with a proximal end and a distal end; in this case, the proximal and distal ends of the first catheter have external diameters sufficient to allow passage through the lumen of the second catheter and combined lengths sufficient for at least the distal part of the first catheter to extend beyond the limits of the distal end of the second catheter, when the first catheter passes through the second catheter; in this case, the internal diameter of the lumen of the second catheter exceeds the external diameter of the distal part of the third catheter; in this case the anti-embolus filter is a series of through holes made in the wall of the principal through lumen on the proximal end of the principal through lumen of the distal part of the first catheter; in this case the length of the conically narrowing proximal end of the principal through lumen of the distal part of the first catheter ranges from 2 cm to 40 cm, and the above-mentioned holes made in the wall of the principal through lumen are not less than 50 μm and not more than 300 μm in diameter; in this case, the end-point of the proximal end of the through lumen of the distal part of the first catheter contains a hole equal in diameter to that of the guidewire that passes through said hole into the principal through lumen of the distal part of the first catheter.

In addition, the proximal part of the first catheter consists of one lumen for filling the balloon, while the distal part of the first catheter takes the form of a two-lumen tube comprising a principal through lumen with an internal diameter sufficient to allow blood to pass through said principal lumen, and the lumen for filling the balloon, which is narrower than the principal through lumen and connects with the previously mentioned lumen for filling the balloon in the proximal part of the first catheter.

In addition, the lumen for filling the balloon in the distal part of the first catheter ends at the place of positioning of the balloon, while the principal through lumen passes through the place of positioning of the balloon and on to the distal end of the distal part of the first catheter.

In addition, the balloon is made of an elastomer material.

Additionally, the distal part of the first catheter is made of radio-opaque material or contains at least one radio-opaque marker.

In addition, the principal through lumen of the distal part of the first catheter has an internal diameter of not less than 1 mm, but not more than 2.5 mm.

In addition, the distal part of the first catheter has an external diameter of not less than 1.2 mm, but not more than 2.8 mm.

Additionally, the proximal end of the lumen for filling the balloon in the proximal part of the first catheter is closed with an elastic material that allows an injection needle to pass through.

In this case, the length of the conically narrowing proximal end of the principal through lumen of the distal part of the first catheter is within a range of 2 cm to 30 cm, preferably to 20 cm.

In this case, the distal end of the principal through lumen of the distal part of the first catheter is closed with an additional anti-embolus filter.

Additionally, the kit contains a third catheter, made in the form of a single-lumen tube, with a proximal end and a distal end; in this case, the proximal and distal parts of the first catheter have external diameters sufficient to allow passage through the lumen of the third catheter and combined length sufficient to allow at least the distal part of the first catheter to extend beyond the limits of the distal end of the third catheter, when the first catheter passes through the third catheter; in this case, the internal diameter of the lumen of the third catheter is equal to the external diameter of the distal part of the first catheter.

In accordance with a fourth embodiment, the kit for preventing distal embolism during endovascular interventions includes the following: a first catheter comprising a proximal part and distal part with a principal through lumen, a lumen for filling the balloon, an inflated balloon which closes the lumen of the right or left common carotid artery and positioned on said distal part from the side of its distal end, and an anti-embolus filter placed on the proximal end of the principal through lumen of the distal part of the first catheter; and a second catheter made in the form of a single-lumen tube with a proximal end and a distal end; in this case, the proximal and distal ends of the first catheter have internal diameters sufficient to allow passage of the second catheter through the lumen and combined lengths sufficient for the distal part at least to extend beyond the limits of the distal end of the second catheter, when the first catheter passes through the second catheter; in this case, the internal diameter of the lumen of the second catheter exceeds that of the external diameter of the distal part of the first catheter; in this case the anti-embolus filter takes the form of a braided, knitted or meshed conical polymer filter the length of which is within a range of 2 cm to 40 cm.

In addition, the proximal part of the first catheter consists of one lumen for filling the balloon, while the distal part of the first catheter takes the form of a two-lumen tube comprising a principal through lumen with an internal diameter sufficient to allow blood to pass through said principal lumen, and the lumen for filling the balloon, which is narrower than the principal through lumen and connects with the previously mentioned lumen for filling the balloon in the proximal part of the first catheter.

In addition, the lumen for filling the balloon in the distal part of the first catheter ends at the place of positioning of the balloon, while the principal through lumen passes through the place of positioning of the balloon and on to the distal end of the distal part of the first catheter.

In this case, the balloon is made of an elastomer material.

In this case, the distal part of the first catheter is made of radio-opaque material or contains at least one radio-opaque marker.

In addition, the principal through lumen of the distal part of the first catheter has an internal diameter of not less than 1 mm, but not more than 2.5 mm.

In addition, the distal part of the first catheter has an external diameter of not less than 1.2 mm, but not more than 2.8 mm.

Also, the proximal end of the lumen for filling the balloon in the proximal part of the first catheter is closed with an elastic material that allows an injection needle to pass through.

In this case, the length of the anti-embolus filter is within a range of 2 cm to 30 cm, preferably to 20 cm.

In this case, the holes in the anti-embolus filter measure from 50 to 300 μm, more preferably to 100 μm.

In addition, the anti-embolus filter contains an additional hole, the diameter of which equals that of the guidewire that passes through said hole into the principal through lumen of the distal part of the first catheter.

In this case, the distal end of the principal through lumen of the distal part of the first catheter is closed with an additional anti-embolus filter.

Additionally, the kit contains a third catheter, made in the form of a single-lumen tube, with a proximal end and a distal end; in this case, the proximal and distal parts of the first catheter have external diameters sufficient to allow passage through the lumen of the third catheter and combined length sufficient to allow at least the distal part of the first catheter to extend beyond the limits of the distal end of the third catheter, when the first catheter passes through the third catheter; in this case, the internal diameter of the lumen of the third catheter is equal to the external diameter of the distal part of the first catheter.

In accordance with a fifth embodiment, the kit for preventing distal embolism during endovascular interventions includes the following: a first catheter comprising a proximal part and a distal part with a principal through lumen, a lumen for filling the balloon, an inflated balloon which closes the lumen of the right or left common carotid artery and is positioned on said distal part from the side of its distal end, and an anti-embolus filter placed on the proximal end of the principal through lumen of the distal part of the first catheter; and a second catheter made in the form of a single-lumen tube with a proximal end and a distal end; in this case the proximal and distal parts of the first catheter have external diameters that allow passage through the lumen of the second catheter, and combined lengths that are sufficient for at least the distal part of the first catheter to extend beyond the limits of the distal end of the second catheter, when the first catheter passes through the second catheter; in this case the internal diameter of the second catheter exceeds the external diameter of the distal part of the first catheter; and the anti-embolus filter takes the form of network that closes the proximal hole in the principal through lumen.

In addition, the proximal part of the first catheter consists of one lumen for filling the balloon, and the distal part of the first catheter takes the form of a two-lumen tube which includes a principal through lumen with an internal diameter sufficient to allow blood to flow through said principal lumen and a lumen for filling the balloon which is narrower than the principal through lumen and connects with the previously mentioned lumen for filling the balloon in the proximal part of the first catheter.

In this case, the lumen for filling the balloon in the distal part of the first catheter ends at the place of positioning of the balloon, while the principal through lumen passes through the place of positioning of the balloon and as far as the distal end of the distal part of the first catheter.

In this case, the balloon is made of an elastomer material.

In addition, the distal part of the first catheter is made from radio-opaque material or contains at least one radio-opaque marker.

In addition, the principal through lumen of the distal part of the first catheter has an internal diameter of not less than 1 mm, but not more than 2.5 mm.

In this case, the distal part of the first catheter has an external diameter of not less than 1.2 mm but not more than 2.8 mm.

Also, the proximal end of the lumen for filling the balloon in the proximal part of the first catheter is closed with an elastic material that allows an injection needle to pass through.

In addition, the angle α of inclination of the above-mentioned network, which covers the proximal hole in the principal through lumen, is within the range 0°<α≤90°.

In this case, the holes in the above-mentioned network, which covers the proximal hole in the principal through lumen, are from 50 to 300 μm, more preferably to 100 μm, in diameter.

Also, the above-mentioned network contains an additional hole equal in diameter to that of the guidewire passing through said hole into the principal through lumen of the distal part of the first catheter.

In addition, the distal end of the principal through lumen of the distal part of the first catheter is closed with an additional anti-embolus filter.

Additionally, the kit contains a third catheter, made in the form of a single-lumen tube, with a proximal end and a distal end; in this case, the proximal and distal parts of the first catheter have external diameters sufficient to allow passage through the lumen of the third catheter and combined length sufficient to allow at least the distal part of the first catheter to extend beyond the limits of the distal end of the third catheter, when the first catheter passes through the third catheter; in this case, the internal diameter of the lumen of the third catheter is equal to the external diameter of the distal part of the first catheter.

In accordance with a sixth embodiment, the kit for preventing distal embolism during endovascular interventions includes the following: a first catheter, comprising a proximal part and a distal part with a principal through lumen, two lumens for filling two balloons, two balloons placed at a distance from each other and inflated, this inflation closing the lumen of the right or left common carotid artery, and an anti-embolus filter placed on the proximal end of the principal through lumen of the distal part of the first catheter; and a second catheter, made in the form of a single-lumen tube with a proximal end and a distal end; in this case the proximal and distal parts of the first catheter have external diameters sufficient to allow passage through the lumen of the second catheter and combined lengths sufficient for at least the distal part of the first catheter to extend beyond the limits of the distal end of the second catheter, when the first catheter passes through the second catheter; in this case the internal diameter of the lumen of the second catheter exceeds the external diameter of the distal part of the first catheter, in which case the anti-embolus filter takes the form of a braided, knitted or meshed conical polymer filter, the length of which is within the range 2 cm to 40 cm.

In addition, the proximal part of the first catheter consists of two lumens for filling two balloons, while the distal part of the first catheter takes the form of a three-lumen tube, which includes a principal through lumen the internal diameter of which allows blood to flow through said through lumen, and two lumens for filling two balloons which are narrower than the principal through lumen and connect with the previously mentioned two lumens for filling two balloons in the proximal part of the first catheter.

In addition, each lumen for filling a balloon in the distal part of the first catheter ends at the place of positioning of the corresponding balloon, and the principal through lumen passes through the place of positioning of the balloons and to the distal end of the distal part of the first catheter.

In this case, the balloons are made of an elastomer material.

In this case, the distal part of the first catheter is made of radio-opaque material or contains at least one radio-opaque marker.

In addition, the principal through lumen of the distal part of the first catheter has an internal diameter of not less than 1 mm, but not more than 2.5 mm.

In addition, the distal part of the first catheter has an external diameter of not less than 1.2 mm, but not more than 2.8 mm.

In this case, the proximal ends of the lumens for filling the balloons in the proximal part of the first catheter are closed with elastic material that allows an injection needle to pass through.

In this case, the length of the anti-embolus filter is within the range 2 cm to 30 cm, preferably to 20 cm.

Also, the holes in the anti-embolus filter measure from 50 to 300 μm, more preferably to 100 μm.

In addition, the anti-embolus filter contains an additional hole, the diameter of which equals that of the guidewire that passes through said hole into the principal through lumen of the distal part of the first catheter.

In this case, the distance between the balloons is 3-4 cm.

In addition, the distal end of the principal through lumen of the distal part of the first catheter is closed with an additional anti-embolus filter.

Additionally, the kit contains a third catheter, made in the form of a single-lumen tube, with a proximal end and a distal end; in this case, the proximal and distal parts of the first catheter have external diameters sufficient to allow passage through the lumen of the third catheter and combined length sufficient to allow at least the distal part of the first catheter to extend beyond the limits of the distal end of the third catheter, when the first catheter passes through the third catheter; in this case, the internal diameter of the lumen of the third catheter is equal to the external diameter of the distal part of the first catheter.

In accordance with a first embodiment for the method for preventing distal embolism during endovascular interventions using one of the above-mentioned devices comprising two catheters, the method consists of stages during which: the femoral artery, radial artery or brachial artery is injected, an introducer is inserted into the common femoral artery, radial artery or brachial artery, and a guidewire is inserted through said introducer into the left or right common carotid artery; the first catheter is inserted through the guidewire via the previously mentioned introducer; inflation is carried out through at least one lumen for filling at least one balloon in the first catheter, and one balloon in said first catheter inflates at least one balloon in said first catheter until at least one of said balloon closes the lumen of the previously mentioned right or left common carotid artery; after completion of the endovascular intervention, a second catheter is placed on said first catheter in the ascending thoracic aorta and the thoracic aortic arch, bringing said second catheter to the inflated balloon of the previously mentioned first catheter and by swiftly introducing a liquid into said second catheter, any emboli remaining in the areas around the balloon of the previously mentioned first catheter in the left or right common carotid artery are flushed into the distal blood flow behind the thoracic aortic arch; said second catheter is removed, deflating at least one balloon in the previously mentioned first catheter; and the previously mentioned first catheter, guidewire and introducer are removed.

In this case, the method uses the device comprising a first catheter comprising one balloon and one lumen for filling the balloon.

Or the device comprising a first catheter comprising two balloons and two lumens for filling balloons is used.

The liquid used to flush the emboli is saline solution.

In this case, the endovascular intervention is an endovascular aortic valve replacement.

In accordance with a second embodiment for the method for preventing distal embolism during endovascular interventions using one of the above-mentioned devices comprising three catheters, the method includes procedures in which: the common femoral artery, radial artery or brachial artery is injected, an introducer is inserted into said common femoral artery, radial artery or brachial artery; a guidewire is introduced via said introducer into the left or right common carotid artery; the first catheter is inserted through the previously mentioned introducer; at least one lumen for filling at least one balloon in the previously mentioned first catheter is inflated until at least one balloon closes the lumen of the previously mentioned right or left common carotid artery; a third catheter is inserted through the introducer into the previously mentioned first catheter as far as the bearing into the balloon of said first catheter; a liquid medical preparation is introduced into said third catheter; after the endovascular intervention is completed, a second catheter is introduced into said first catheter in the ascending aorta and in the aortic arch; said second catheter is introduced to the inflated balloon in the previously mentioned first catheter and by swiftly introducing a liquid into said second catheter, any emboli remaining in the areas around the balloon of the previously mentioned first catheter in the left or right common carotid artery are flushed into the distal blood flow behind the thoracic aortic arch; said second catheter is removed, deflating at least one balloon in the previously mentioned first catheter; and the previously mentioned first catheter, guidewire and introducer are removed.

In this case the device comprising a first catheter comprising one balloon and one lumen for inflating the balloon is used.

Or the device comprising a first catheter comprising two balloons and two lumens for filling balloons is used.

The liquid used to flush the emboli is saline solution.

In this case, the endovascular intervention is an endovascular aortic valve replacement.

In accordance with a seventh embodiment, the kit for preventing distal embolism during endovascular interventions includes the following: a first catheter, comprising a proximal part and a distal part with a principal through lumen, two lumens for filling two balloons, two balloons placed at a distance from each other and inflated, one of which, the distal, is inflated, this inflation closing the lumen of the right or left common carotid artery, and the other, the proximal, is inflated to close the lumen of the right or left common subclavian artery; and a second catheter, made in the form of a single-lumen tube with a proximal end and a distal end; in this case the proximal and distal parts of the first catheter have external diameters sufficient to allow passage through the lumen of the second catheter and combined lengths sufficient for at least the distal part of the first catheter to extend beyond the limits of the distal end of the second catheter, when the first catheter passes through the second catheter; in this case the internal diameter of the lumen of the second catheter exceeds the external diameter of the distal part of the first catheter, in which case the part of the distal part of the first catheter placed between the balloons allows blood to flow through said principal lumen but blocks the passage of emboli through it.

In addition, the proximal part of the first catheter consists of two lumens for filling two balloons, and the distal part of the first catheter takes the form of a three-lumen tube comprising a principal through lumen the internal diameter of which allows blood to flow through said principal lumen, and two lumens for filling two balloons which are narrower than the principal through lumen and connected with the previously mentioned two lumens for filling the two balloons in the proximal part of the first catheter.

In addition, each lumen for filling a balloon in the distal part of the first catheter ends at the place of positioning of the corresponding balloon, while the principal through lumen passes through the place of positioning of the balloons and as far as the distal end of the distal part of the first catheter.

In this case, the balloons are made of an elastomer material.

In this case, the distal part of the first catheter is made of radio-opaque material or contains at least one radio-opaque marker.

In this case, the principal through lumen of the distal part of the first catheter has an internal diameter of not less than 1 mm, but not more than 2.5 mm.

In addition, the distal part of the first catheter has an external diameter of not less than 1.2 mm, but not more than 2.8 mm.

Additionally, the proximal ends of the lumens for filling balloons in the proximal part of the first catheter are closed with elastic material that allows an injection needle to pass through.

In addition to the above, the length of the part in the distal part of the first catheter, placed between the balloons, is 3-4 cm.

In this case, the wall of the principal through lumen of the part of the distal part in the first catheter positioned between the balloons contains holes.

The above-mentioned holes made in the wall of the principal through lumen are 50 to 300 μm and more preferably to 100 μm in diameter.

Additionally, the part of the distal part of the first catheter positioned between the balloons takes the form of a braided, knitted or meshed polymer anti-embolus filter.

In this case, the holes in the anti-embolus filter measure from 50 to 300 μm and more preferably to 100 μm.

In accordance with one appearance, the proximal end of the principal through lumen of the distal part of the first catheter is kept wholly open.

In accordance with another appearance, the anti-embolus filter is positioned on the proximal end of the principal through lumen of the distal part of the first catheter.

In this case, the anti-embolus filter placed on the proximal end of the principal through lumen of the distal part of the first catheter takes the form of a braided, knitted or meshed conical polymer filter, the length of which is the range 2 to 40 cm, preferably to 30 cm and more preferably to 20 cm.

In this case, the holes in the anti-embolus filter placed on the proximal end of the principal through lumen of the distal part of the first catheter measure from 50 to 300 μm, more preferably to 100 μm.

Also, the anti-embolus filter placed on the proximal end of the principal through lumen of the distal part of the first catheter has an additional hole equal in diameter to that of the guidewire passing through said hole into the principal through lumen of the distal part of the first catheter.

According to one other appearance, the proximal end of the principal through lumen of the distal part of the first catheter is closed with a hole equal in diameter to that of the guidewire passing through said hole into the principal through lumen of the distal part of the first catheter.

Additionally, the distal end of the principal through lumen of the distal part of the first catheter is closed with an additional anti-embolus filter.

In some cases, the kit additionally contains a third catheter made in the form of a single-lumen tube with a proximal end and distal end; in this case the proximal and distal parts of the first catheter have external diameters sufficient to allow the passage of the third catheter through the lumen and combined lengths sufficient for at least the distal part of the first catheter to extend beyond the limits of the distal end of the third catheter, when the first catheter passes through the third catheter; in this case, the internal diameter of the lumen of the third catheter is essentially equal to the external diameter of the distal part of the first catheter.

In accordance with an eighth embodiment, the kit for preventing distal embolism during endovascular interventions includes the following: a first catheter, comprising a proximal part and a distal part with a principal through lumen, two lumens for filling two balloons, two balloons placed at a distance from each other and inflated, one of which, the distal, is inflated, this inflation closing the lumen of the right or left common carotid artery, and the other, the proximal, is inflated to close the lumen of the right or left common subclavian artery; and an anti-embolus filter placed on the proximal end of the principal through lumen of the first catheter; and a second catheter made in the form of a single-lumen tube that has a proximal end and a distal end; in this case the proximal and distal parts of the first catheter have external diameters sufficient to allow passage through the lumen of the second catheter and combined lengths sufficient for at least the distal part of the first catheter to extend beyond the limits of the distal end of the second catheter, when the first catheter passes through the second catheter; in this case the internal diameter of the lumen of the second catheter exceeds the external diameter of the distal part of the first catheter, in which case the part of the distal part of the first catheter placed between the balloons allows blood to flow through said principal lumen but blocks the passage of emboli through it.

In addition, the proximal part of the first catheter consists of two lumens for filling two balloons, while the distal part of the first catheter takes the form of a three-lumen tube comprising a principal through lumen with diameter sufficient to allow blood to flow through said principal lumen and two lumens for filling two balloons, these being narrower than the principal through lumen and connected with the previously mentioned two lumens for filling two balloons on the proximal part of the first catheter.

In addition, each lumen for filling a balloon in the distal part of the first catheter ends at the place of positioning of the corresponding balloon, while the principal through lumen passes through the place of positioning of the balloons and as far as the distal end of the distal part of the first catheter.

In this case, the balloons are made of an elastomer material.

In this case, the distal part of the first catheter is made of radio-opaque material or contains at least one radio-opaque marker.

In addition, the principal through lumen of the distal part of the first catheter has an internal diameter of not less than 1 mm, but not more than 2.5 mm.

In addition, the distal part of the first catheter has an external diameter of not less than 1.2 mm, but not more than 2.8 mm.

In this case, the proximal ends of the lumens for filling the balloons in the proximal part of the first catheter are closed with elastic material that allows an injection needle to pass through.

In this case, the length of the distal part of the first catheter, positioned between the balloons, is 3-4 cm.

In accordance with one appearance, the wall of the principal through lumen of the part of the distal part of the first catheter located between the balloons contains holes.

In this case, the previously mentioned holes made in the wall of the principal lumen are from 50 to 300 μm, more preferably to 100 μm, in diameter,

In accordance with another appearance, part of the distal part of the first catheter, positioned between the balloons, takes the form of a braided, knitted or meshed polymer anti-embolus filter.

In this case, the holes in the previously mentioned anti-embolus filter, placed in the part of the distal part of the first catheter located between the balloons, are 50 to 300 μm, more preferably to 100 μm, in diameter.

Also, the anti-embolus filter placed on the proximal end of the principal through lumen of the distal part of the first catheter takes the form of a braided, knitted or meshed conical polymer filter, the length of which is in the range 2 cm to 40 cm, preferably to 30 cm, more preferably to 20 cm.

In this case, the holes in the anti-embolus filter placed on the proximal end of the principal through lumen of the distal part of the first catheter measure 50 to 300 μm, more preferably to 100 μm.

In addition, the anti-embolus filter placed on the proximal end of the principal through lumen of the distal part of the first catheter contains an additional hole equal in diameter to that of the guidewire passing through said hole into the principal through lumen of the distal part of the first catheter.

In this case, the distal end of the principal through lumen of the distal part of the first catheter is closed by an additional anti-embolus filter.

Additionally, the kit contains a third catheter, made in the form of a single-lumen tube, with a proximal end and a distal end; in this case, the proximal and distal parts of the first catheter have external diameters sufficient to allow passage through the lumen of the third catheter and combined length sufficient to allow at least the distal part of the first catheter to extend beyond the limits of the distal end of the third catheter, when the first catheter passes through the third catheter; in this case, the internal diameter of the lumen of the third catheter is equal to the external diameter of the distal part of the first catheter.

In accordance with a ninth embodiment, the kit for preventing distal embolism during endovascular interventions includes the following: a first catheter, comprising a proximal part and a distal part with a principal through lumen, two lumens for filling two balloons, two balloons placed at a distance from each other and inflated, one of which, the distal, is inflated, this inflation closing the lumen of the right or left common carotid artery, and the other, the proximal, is inflated to close the lumen of the right or left common subclavian artery; and a second catheter made in the form of a single-lumen tube that has a proximal end and a distal end; in this case the proximal and distal parts of the first catheter have external diameters sufficient to allow passage through the lumen of the second catheter and combined lengths sufficient for at least the distal part of the first catheter to extend beyond the limits of the distal end of the second catheter, when the first catheter passes through the second catheter; in this case the internal diameter of the lumen of the second catheter exceeds the external diameter of the distal part of the first catheter, in which case the part of the distal part of the first catheter placed between the balloons allows blood to flow through said principal lumen but blocks the passage of emboli through it; in this case the proximal end of the principal through lumen of the distal part of the first catheter is closed with a hole equal in diameter to that of the guidewire passing through said hole into the principal through lumen of the distal part of the first catheter.

In addition, the proximal part of the first catheter consists of two lumens for filling two balloons, and the distal part of the first catheter takes the form of a three-lumen tube comprising a principal through lumen the internal diameter of which allows blood to flow through said principal lumen, and two lumens for filling two balloons which are narrower than the principal through lumen and connected with the previously mentioned two lumens for filling the two balloons in the proximal part of the first catheter.

In addition, each lumen for filling a balloon in the distal part of the first catheter ends at the place of positioning of the corresponding balloon, while the principal through lumen passes through the place of positioning of the balloons and as far as the distal end of the distal part of the first catheter.

In this case, the balloons are made of an elastomer material.

In this case, the distal part of the first catheter is made of radio-opaque material or contains at least one radio-opaque marker.

In addition, the principal through lumen of the distal part of the first catheter has an internal diameter of not less than 1 mm, but not more than 2.5 mm.

In addition, the distal part of the first catheter has an external diameter of not less than 1.2 mm, but not more than 2.8 mm.

In this case, the proximal ends of the lumens for filling the balloons in the proximal part of the first catheter are closed with elastic material that allows an injection needle to pass through.

In this case, the length of the distal part of the first catheter, positioned between the balloons, is 3-4 cm.

In accordance with one appearance, the wall of the principal through lumen of the part of the distal part of the first catheter located between the balloons contains holes.

In this case, the previously mentioned holes made in the wall of the principal lumen are from 50 to 300 μm, more preferably to 100 μm, in diameter,

In accordance with another appearance, part of the distal part of the first catheter, positioned between the balloons, takes the form of a braided, knitted or meshed polymer anti-embolus filter.

In this case, the holes in the previously mentioned anti-embolus filter placed in the part of the distal part of the first catheter placed between the balloons measure 50 to 300 μm, more preferably to 100 μm, in diameter.

In addition, the distal end of the principal through lumen of the distal part of the first catheter is closed with an additional anti-embolus filter.

In accordance with the third embodiment for the method for preventing distal embolism during endovascular interventions using one of the above-mentioned devices comprising two catheters, the method includes stages in which: the radial artery or brachial artery is injected, an introducer is inserted into said radial artery or brachial artery; a guidewire is inserted via said introducer into the left or right common carotid artery; the first catheter is passed along said guidewire through the previously mentioned introducer so that the distal balloon is installed in the left or right common carotid artery respectively and the proximal balloon is installed in the left or right subclavian artery; through one lumen for filling the balloon, first the distal balloon of said first catheter is inflated until the distal balloon closes the lumen of the previously mentioned left or right common carotid artery respectively, and then the proximal balloon of said first catheter is inflated through the other lumen to fill the balloon until the proximal balloon closes the lumen of the previously mentioned left or right subclavian artery; after completion of the endovascular intervention, in the ascending thoracic aorta and the thoracic aortic arch, a second catheter is put on said first catheter; deflating the proximal balloon of the previously mentioned first catheter; the previously mentioned second catheter is brought to the inflated distal balloon of the previously mentioned first catheter and the swift introduction of a liquid substance into said second catheter leads to flushing into the distal blood flow behind the thoracic aortic arch of any emboli remaining in the spaces around the distal balloon of the previously mentioned first catheter in the left or right common carotid arteries; said second catheter is removed, deflating the distal balloon of the previously mentioned first catheter; and the previously mentioned first catheter, guidewire and introducer are removed.

In this case, the liquid used for flushing the emboli is saline solution.

In this case, the endovascular intervention is an endovascular aortic valve replacement.

In accordance with the forth embodiment for the method for preventing distal embolism during endovascular interventions using one of the above-mentioned devices comprising two catheters, the method includes stages in which: the radial artery or brachial artery is injected, an introducer is inserted into said radial artery or brachial artery; a guidewire is inserted via said introducer into the left or right common carotid artery; the first catheter is passed along said guidewire through the previously mentioned introducer so that the distal balloon is installed in the left or right common carotid artery respectively, and the proximal balloon is installed in the left or right subclavian artery; through one lumen for filling the balloon, first the distal balloon of said first catheter is inflated until the distal balloon closes the lumen of the previously mentioned left or right common carotid artery respectively, and then the proximal balloon of said balloon is inflated through another lumen to fill the balloon until the proximal balloon closes the lumen of the previously mentioned left or right subclavian artery; the third catheter is through the introducer on the previously mentioned first catheter from the proximal end, the third catheter is introduced, deflating the proximal balloon of the previously mentioned first catheter; the previously mentioned third catheter is moved forward fully into the distal balloon of said first catheter; a liquid medical preparation is injected into said third catheter; said third catheter is removed; the proximal balloon of the previously mentioned first catheter is inflated until the proximal balloon closes the lumen of the previously mentioned left or right subclavian artery; after completion of the endovascular intervention, in the ascending thoracic aorta and the thoracic aortic arch, a second catheter is put onto said first catheter, deflating the proximal balloon of the previously mentioned first catheter; the previously mentioned second catheter is brought to the inflated distal balloon of the previously mentioned first catheter and the swift introduction of a liquid substance into said second catheter leads to flushing into the distal blood flow behind the thoracic aortic arch of any emboli remaining in the spaces around the distal balloon of the previously mentioned first catheter in the left or right common carotid arteries; said second catheter is removed, deflating the distal balloon of the previously mentioned first catheter; the previously mentioned first catheter, guidewire and introducer are removed.

In this case, the liquid used for flushing the emboli is saline solution.

In this case, the endovascular intervention is an endovascular aortic valve replacement.

### BRIEF DESCRIPTION OF THE DRAWINGS

The essence of the proposed group of inventions is shown in the drawings:

FIG. 1A: embodiment for first catheter of proposed kit with distal part, cylindrical throughout its length, one balloon and holes located at distance of 20-70 cm from the balloon.

FIG. 1B: embodiment for first catheter of proposed kit with distal part, conically narrowing at its proximal end, with one balloon and holes.

FIG. 1C: embodiment for first catheter of proposed kit with distal part, with one balloon and braided (or knitted or meshed) conical polymer anti-embolus filter on its proximal end.

FIG. 1D: embodiment for first catheter of proposed kit with distal part, with one balloon and network closing the proximal hold in the principal through lumen of the distal part.

FIG. 2: embodiment for second and third catheter in proposed kit.

FIG. 3A: embodiment for first catheter of proposed kit with distal part, with two balloons and a braided, knitted or meshed conical polymer anti-embolus filter on its proximal end.

FIG. 3B: embodiment for first catheter of proposed kit with distal part, with two balloons, a braided (or knitted or meshed) conical polymer anti-embolus filter on its proximal end and an additional anti-embolus filter on its distal end.

FIG. 4A: Illustration of positioning of first catheter with one balloon and anti-embolus filter on proximal end of distal part, according to method for using the kit declared.

FIG. 4B: Illustration of stage of use of third catheter according to method for using the kit declared.

FIG. 4C: Illustration of stage of use of second catheter according to method for using the kit declared.

FIG. 5A: Illustration of positioning of first catheter with two balloons and braided (or knitted or meshed) conical anti-embolus filter on proximal end of distal part, according to method for using the kit declared.

FIG. 5B: Illustration of stage of use of third catheter according to method for using the kit declared.

FIG. 5C: Illustration of stage of use of second catheter according to method for using the kit declared.

FIG. 6A: embodiment for first catheter of proposed kit with distal part, with two balloons and a part positioned between them which allows blood to flow through the distal part but blocks passage of emboli through that part.

FIG. 6B: embodiment for first catheter of proposed kit with distal part, with two balloons, a part positioned between them which allows blood to flow through the distal part but blocks passage of emboli through that part, an anti-embolus filter on its proximal part and an additional anti-embolus filter on its distal end.

FIG. 6C: embodiment for first catheter of proposed kit with distal part, with two balloons, a part positioned between them which allows blood to flow through the distal part but blocks passage of emboli through that part, a closed proximal end and an additional anti-embolus filter on its distal end.

FIG. 7A: Illustration of positioning of first catheter with two balloons, a part positioned between them which allows blood to flow through the distal part but blocks passage of emboli through that part, an anti-embolus filter on its proximal end and an additional anti-embolus filter on its distal end.

FIG. 7B: Illustration of stage of use of third catheter according to method for using the kit declared.

FIG. 7C: Illustration of stage of use of second catheter according to method for using the kit declared.

The drawings are not strictly to scale, and details not necessary for understanding the invention may be omitted. Other elements that are at least equivalent to each other or at least have equivalent functions to each other are referred to using the same figure.

### DESCRIPTION OF REFERENCE TO PARTS

1—first catheter;
2—second catheter;
3—third catheter;
4—distal part of first catheter;
5—proximal part of first catheter;
6—balloon;
7—proximal end of distal part of first catheter;
8—holes;
9—braided (knitted or meshed) conical filter;
10—network closing the proximal hole of the principal through lumen of distal part of first catheter;
11—additional anti-embolus filter closing the distal end of the principal through lumen of the distal part of the first catheter;
12—part of distal part of first catheter that allows blood to flow through the distal part but blocks emboli from passing through;
13—closed proximal end of distal part of first catheter;
14—carotid artery;
15—subclavian artery.

### DETAILED DESCRIPTION

Here follows a more detailed description of one of more options (embodiments) for implementation, examples of which are shown in the drawings. Each example and option of implementation are shown for explanation and do not suggest any limitation. For example, the particular features illustrated or described as part of one option for implementation may be used with another variation of implementation to produce an additional option for implementation. The intention is that the claims cover these and other modifications and options (embodiments) that are within the scope and the essence of the invention.

In the following description, the term “proximal” refers to parts of constructions from the proposed kit and to an organ or part thereof located closer to the access point (or to the healthcare professional) during implementation of the proposed methods. Correspondingly, the term “distal” refers to parts of constructions from the proposed kit and to an organ or part thereof located further away from the access point (or from the healthcare professional) during implementation of the proposed methods.

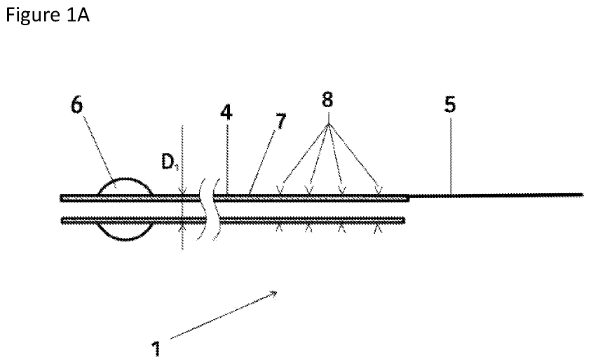

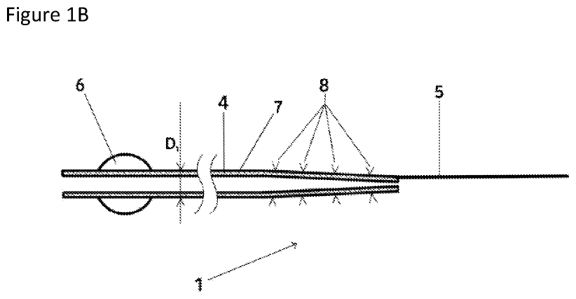

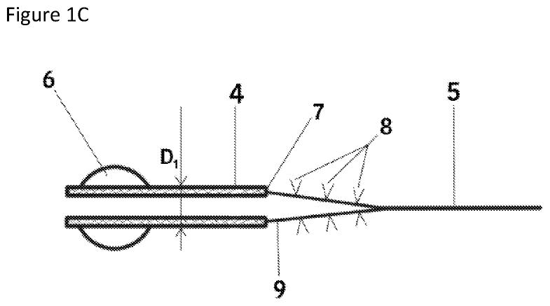

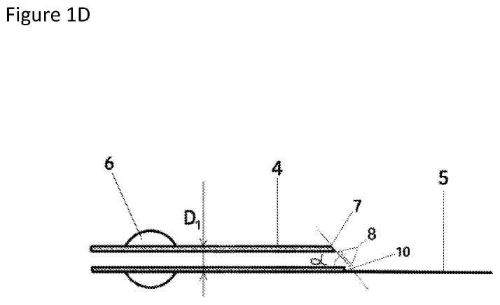

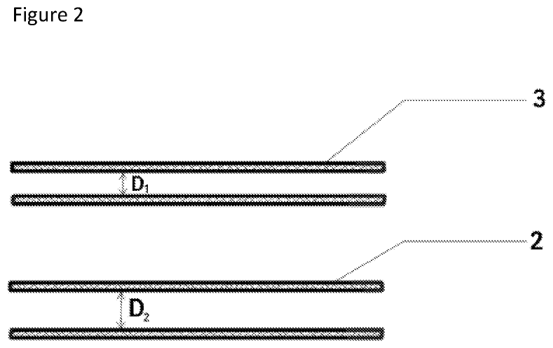

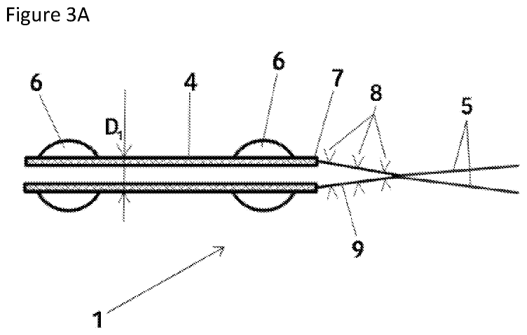

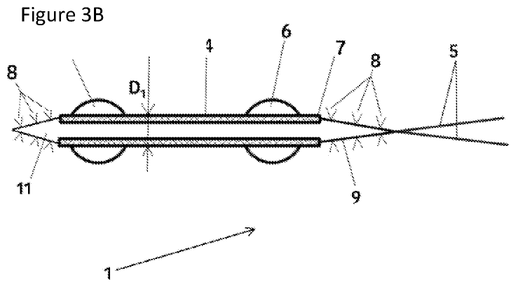

In accordance with one principal aspect of the invention, the kit for preventing distal embolism during endovascular interventions includes a first catheter (1) and a second catheter (2). In turn, as illustrated on FIGS. 1A-1D and 3A-3B, the first catheter (1) contains a proximal part (5) and a distal part (4). The distal part (4) has a principal through lumen (not shown on the drawings), at least one lumen (not shown on the drawings) for filling at least one balloon, one balloon (6) that is inflated and thus closes the lumen of the right or left common carotid artery, and a proximal end (7) which allows blood to flow through said principal lumen but blocks the passage of emboli through it. In turn, as illustrated in FIG. 2, the second catheter (2) takes the form of a single-lumen tube with a proximal end and a distal end. The first and second catheters are produced taking account of each other's dimensions: the proximal and distal parts (5, 4) of the first catheter (1) have external diameters sufficient to allow the passage through the lumen of the second catheter (2), and combined lengths sufficient to allow at least the distal part (4) of the first catheter (1) to extend beyond the limits of the distal end of the second catheter (2), when the first catheter (1) passes through the second catheter (2); in addition, the internal diameter (D₂) of the lumen of the second catheter (2) exceeds the external diameter (D₁) of the distal part (4) of the first catheter (1): D₂>D₁.

The proximal part (5) of the first catheter (1) consists of at least one lumen for filling at least one balloon (FIGS. 1A-1D, 3A-3B). The distal part (4) of the first catheter (1) takes the form of at least one two-lumen tube, which comprises: a principal through lumen (not shown on drawings) the internal diameter of which allows blood to flow through said principal lumen and at least one lumen (not shown on drawings) for filling at least one balloon, this lumen being narrower than the principal through lumen and connected to the previously mentioned at least one lumen for filling at least one balloon in the proximal part (5) of the first catheter (1). At least one lumen (not shown on drawing) for filling at least one balloon (6) in the distal part (4) of the first catheter (1) ends in the place of positioning of at least one balloon (6), while the principal through lumen (not shown on drawing) passes through the place of positioning of at least one balloon (6) and as far as the distal end of the distal part (4) of the first catheter (1).

At least one balloon (6) may be made from an elastomer material such as latex, silicone, polyamide-based polymer or polyurethane, thermoplastic elastomer C-Flex™, ChronoThane™, Santoprene™, or similar material.

The distal part (4) of the first catheter (1) may be made of radio-opaque material such as radio-opaque polyurethane, polyethylene or propylene, or may contain at least one radio-opaque marker. At least one marker may be made by applying a radio-opaque substance as a covering to the surface of the wall of the distal part of the first catheter, consisting for example of polyurethane or nylon. This can be carried out by rolling, spraying, printing, stamping, painting, gluing, or otherwise applying a radio-opaque substance to the distal wall of the first catheter. Also, at least one marker may be created during manufacture of the distal wall of the first catheter. Examples of radio-opaque substances include, but are not limited to, tungsten powder, tantalum, bismuth, bismuth trioxide, bismuth oxychloride, bismuth carbonate, other bismuth compounds, barium sulphate, tin-plate, silver, silver compounds, oxides of rare earths and many other substances widely used to absorb X-rays. The quantity used may depend on the required level of radio-opacity.

In accordance with one option for producing the kit for preventing distal embolism during endovascular interventions, the distal part (4) of the first catheter (1), as shown on FIGS. 1A-1D, may have a principal through lumen (not shown on drawings), one lumen (not shown on drawings) for filling one balloon, and a balloon (6) inflated to close the lumen of the right or left common carotid artery and located on the side of the distal end on said distal part (4), while the proximal part (5) of the first catheter (1) consists of one lumen for filling one balloon.

On the proximal side (7) of the principal through lumen of the distal part (4) of the first catheter (1) is an anti-embolus filter.

In accordance with the option for producing the first catheter shown in FIG. 1A, the anti-embolus filter may be a series of through holes (8) made in the wall of the principal through lumen on the proximal end (7) of the principal through lumen of the distal side (4) of the first catheter (1), in which case the principal through lumen of the distal part (4) of the first catheter (1) is cylindrical throughout its length; in this case the above-mentioned holes (8) are positioned not less than 20 cm and not more than 70 cm from the balloon and range from 50 to 300 μm, more preferably to 100 μm, in diameter. At a distance of at least 20 cm, the holes pass into the descending thoracic aorta, and at 70 cm at least, they do not go out. The diameter of the hole ranges from 50 μm (to allow all components of blood to pass) to 300 μm (to prevent passage of functionally significant emboli). Preferably to 100 μm means the average values.

In accordance with the option for the first catheter shown in FIG. 1B, the anti-embolus filter may include a series of through holes (8), made in the wall of the principal through lumen on the proximal end (7) of the principal through lumen of the distal part (4) of the first catheter (1); in this case the principal through lumen of the distal part (4) of the first catheter (1) narrows conically in its proximal end (7), and the above-mentioned holes (8) have a diameter ranging from 50 to 300 mm, more preferably to 100 mm, and the length of the conically narrowing proximal end (7) of the principal through lumen of the distal part (4) of the first catheter (1) is within the range 2 cm to 40 cm, preferably to 30 cm, more preferably to 20 cm. The length of the clinically narrowing end is determined by functional requirements for the catheter and ease of handling.

In accordance with the option for the first catheter shown in FIG. 1C, the anti-embolus filter may take the form of a braided, knitted or meshed conical polymer filter (9), for example a filter produced in a way similar to that known from the description to patent RU 2597371, the length of said conical filter being within the range of 2 cm to 40 cm, preferably to 30 cm and more preferably to 20 cm, in which case the holes in said anti-embolus filter may be of the previously mentioned size, 50 to 300 μm and more preferably to 100 μm.

In accordance with the option for the first catheter shown in FIG. 1D, the anti-embolus filter may take the form of a network (10) that closes the proximal hole of the principal through lumen; in this case the angle α of inclination of the above-mentioned network (10), closing the proximal hole in the principal through lumen is within the range 0°<α≤90°.

Also admissible is an option (not shown in the drawings) according to which the anti-embolus filter takes the form of a porous membrane that closes the proximal hole in the principal through lumen.

In accordance with another option for producing the kit for preventing distal embolism during endovascular interventions, the distal part (4) of the first catheter (1), as shown in FIG. 3A-3B, may have a principal through lumen (not shown in the drawings), two lumens (not shown in the drawings) for filling two balloons and two balloons positioned at a distance from each other (6), and inflated, thus closing the lumen of the right or left common carotid artery, with the proximal part (5) of the first catheter (1) consisting of two lumens for filling two balloons.

Also, the above-mentioned first catheter (1) on the proximal end (7) of the principal through lumen of the distal part (4) of the first catheter (1) contains an anti-embolus filter.

In accordance with the option for producing the first catheter (1) shown in FIG. 3A, the anti-embolus filter may take the form of a braided or knitted or meshed conical polymer filter (9) similar to that mentioned above.

In addition to the other anti-embolus filter for said option for the third catheter (1), it may also take the form of a network (10) that closes the proximal hole of the principal through lumen; in this case the angle α of inclination of the above-mentioned network that closes the proximal hole in the principal through lumen is in the range 0°<α≤90°, while the anti-embolus filter may take the form of a porous membrane (not shown in the drawings), which covers the proximal opening of the principal through lumen.

Preferably, the distance between the balloons (6) should be 3-4 cm to ensure function of the catheter in the different anatomical options and vessel lengths.

For a series of options for the kit for preventing distal embolism during endovascular interventions, the end-point of the proximal end (7) of the principal through lumen of the distal part (4) of the first catheter (1) contains a hole (not shown in the drawings), equal in diameter to the diameter of the guidewire passed during introduction of the first catheter (1) into the vessel through said hole into the principal through lumen of the distal part (4) of the first catheter (1).

In addition, in all the above-mentioned options for producing the kit, the principal through lumen of the distal part (4) of the first catheter (1) has an internal diameter of not less than 1 mm but not more than 2.5 mm; these are the “working” dimensions of standard endovascular catheters. In addition, the distal part (4) of the first catheter (1) has an external diameter (D₁) of not less than 1.2 mm but not more than 2.8 mm (dimensions of standard catheters).

It is also advisable for the proximal end of at least one lumen for filling at least one balloon in the proximal part of the first catheter to be closed with elastic material made to allow an injection needle to pass through, by analogy with the generally known infusion (injection) ports.

All of the above options for the first catheters, not limited by the option illustrated on FIG. 3B, may, for more effective prevention of embolism, contain an additional anti-embolus filter (11) which closes the distal end of the principal through lumen of the distal part (4) of the first catheter (1).

And finally, as an additional element over and above the first and second catheter, the kit for preventing distal embolism during endovascular interventions may contain a third catheter (3) (FIG. 2), designed for introducing a liquid medicinal preparation (such as saline solution) and taking the form of a single-lumen tube with a proximal end and distal end.

The first and third catheters are made taking account of the following dimensions of each: the proximal and distal parts (5, 4) of the first catheter (1) have external diameters sufficient to allow passage through the lumen of a third catheter (3) and combined lengths sufficient to allow at least the distal part of the first catheter (1) to extend beyond the limits of the distal end of the third catheter (3) when the first catheter (1) passes through the third catheter (3); in addition the internal diameter (D₃) of the lumen of the third catheter (3) is essentially equal to the external diameter (D₁) of the distal part of the first catheter (1): D₃=D₁.

The above-mentioned options for the kit are used as follows:

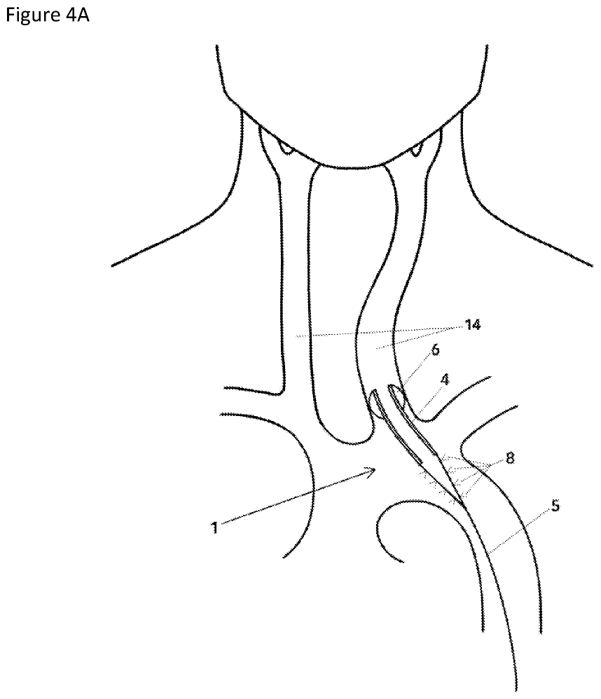

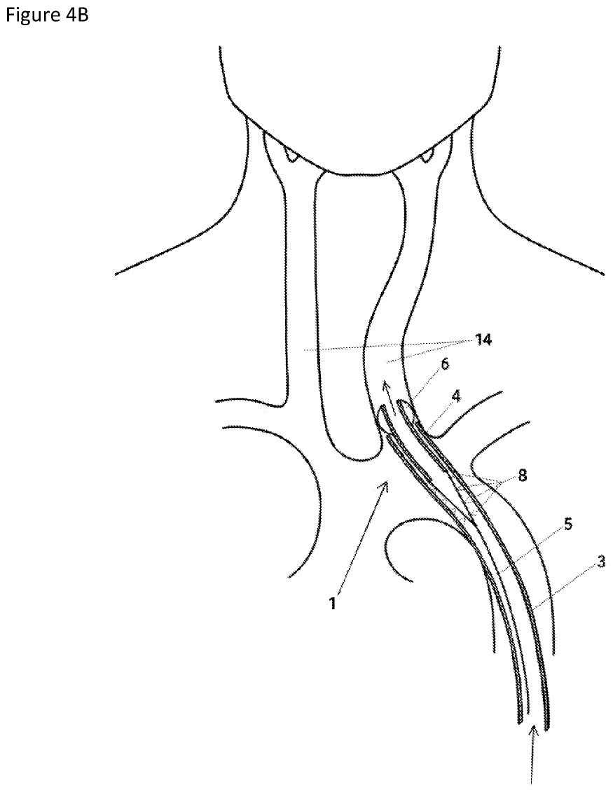

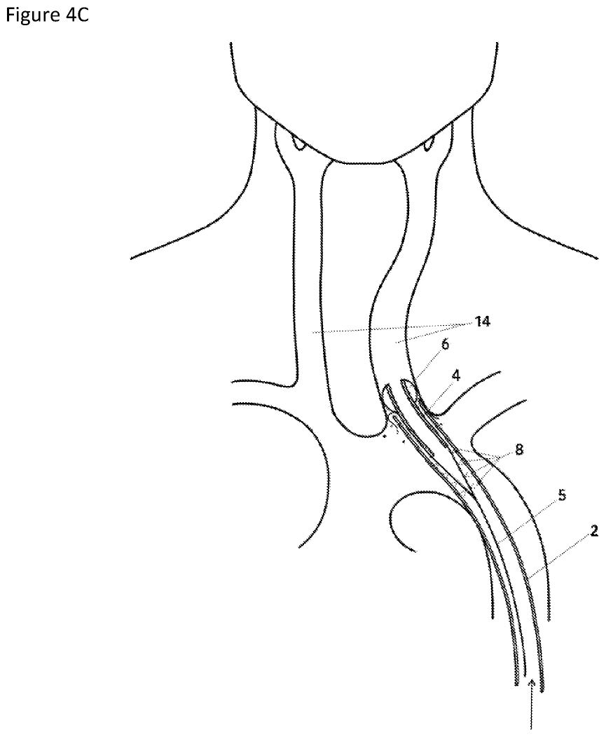

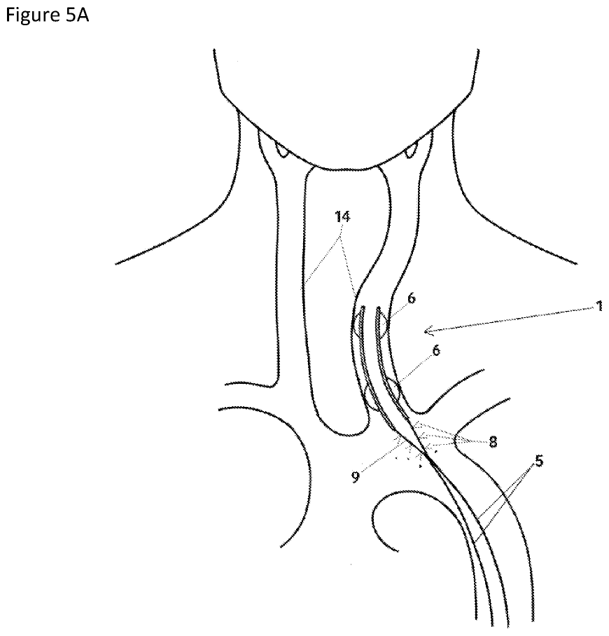

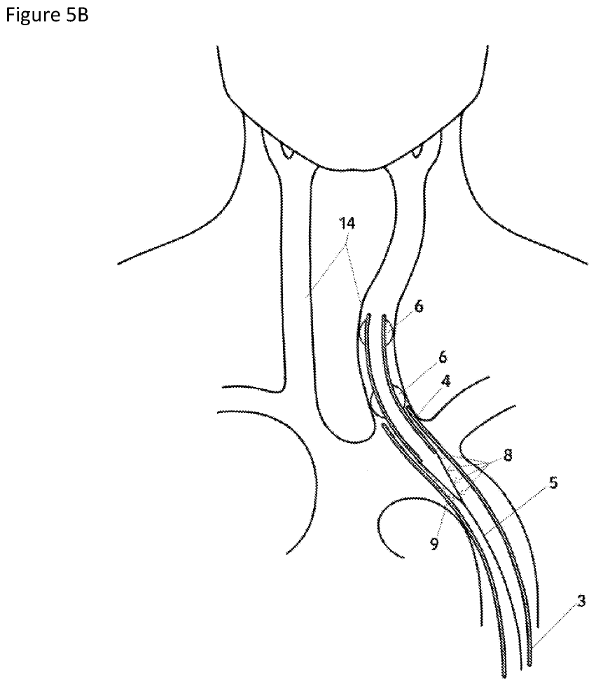

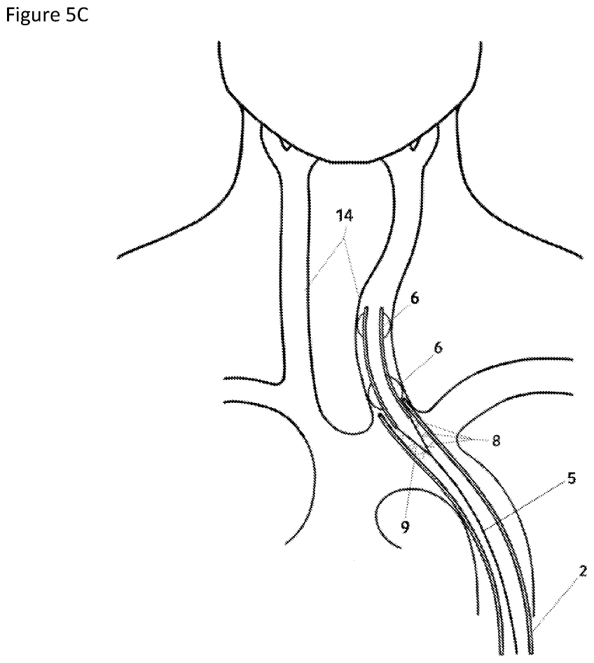

Using the generally known methods, the common femoral artery, the radial artery or the brachial artery is injected. The introducer is then inserted into said common femoral artery, radial artery or brachial artery. Through said introducer, the guidewire is then inserted into the left or right common carotid artery (14). The first catheter (1) is then inserted along said guidewire, through the previously mentioned introducer. Through at least one lumen for filling at least one balloon in said first catheter, at least one balloon (6) in said first catheter (1) is inflated until, using at least one of said balloons (6), the lumen of the previously mentioned right or left common carotid artery (14) is closed (FIGS. 4A, 5A). A third catheter (3) is inserted through the introducer into the previously mentioned first catheter (1) from the proximal end all the way into the balloon (6) of said first catheter (1) (obviously if the first catheter (1) is used with one balloon (6), the third catheter (3) will be inserted through to the single balloon (6) in the first catheter (1) (FIG. 4B); if the first catheter (1) is used with two balloons (6), the third catheter (3) will be inserted all the way into the proximal balloon (6) of the first catheter (1) (FIG. 5B). A liquid medicinal preparation, such as saline solution, is then introduced into said third catheter (3) (FIGS. 4B, 5B). After all the medicinal preparation has been introduced, the third catheter (3) is removed. After completion of the endovascular intervention, the second catheter (2) is introduced into said first catheter (1) in the ascending thoracic aorta and the thoracic aortic arch. Said second catheter (2) is inserted all the way to the inflated balloon (6) in the previously mentioned first catheter (1) (obviously, if the first catheter (1) is used with one balloon (6), the second catheter will be inserted all the way to the single balloon (6) in the first catheter (1) (FIG. 4C); if the first catheter (1) is used with two balloons (6), the second catheter (2) will be inserted all the way to the proximal balloon (6) in the first catheter (1) (FIG. 5B)) and with swift introduction of the liquid substance such as saline solution. Any emboli remaining in the spaces around the balloon (6) of the previously mentioned first catheter (1) in the left or right common carotid artery (14), are flushed into said second catheter (2) and into the distal blood flow behind the thoracic aortic arch. The second catheter (2) is then removed, the at least one balloon (6) in the previously mentioned first catheter (1) is deflated, and the previously mentioned first catheter (1), guidewire and introducer are removed.

To prevent distal embolism, and in particular embolisms in the cerebral vessels, using said method, it is possible, as stated above, to use both a device comprising a first catheter (1) comprising one balloon (6) and one lumen (not shown in the drawings) for filling the balloon (FIGS. 1A-1D), and a device including a first catheter (1) comprising two balloons (6) and two lumens (not shown in the drawings) for filling the balloons (FIGS. 3A-3B).

It should also be noted that if there is no requirement to introduce medicinal preparations into the distal area of the endovascular intervention, the device used shall consist only of a first and second catheter, and therefore these stages of the method, namely the application of the third catheter to the first catheter, the introduction of the preparation and the removal of the third catheter, will not be carried out.

In addition, it should be noted that the above-mentioned devices may be used to prevent cerebral embolism during aortic valve replacement procedures and insertion of artificial ascending aorta and thoracic aortic arch. It is clear to the specialist that in order to provide more effective protection against embolism, a number of devices can be used simultaneously, such as two, in which case the first catheter from the first device is inserted in the right or left common carotid artery and, correspondingly, the first catheter from the other (second) device is inserted in the left or right common carotid artery.

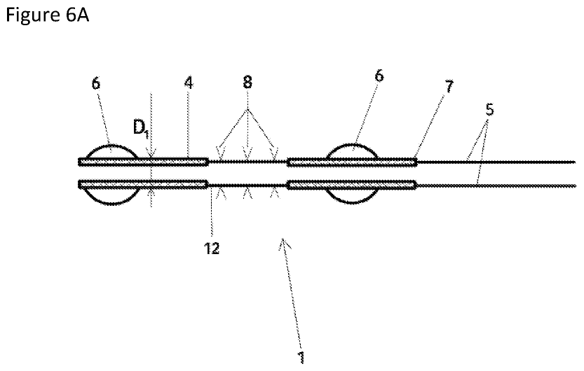

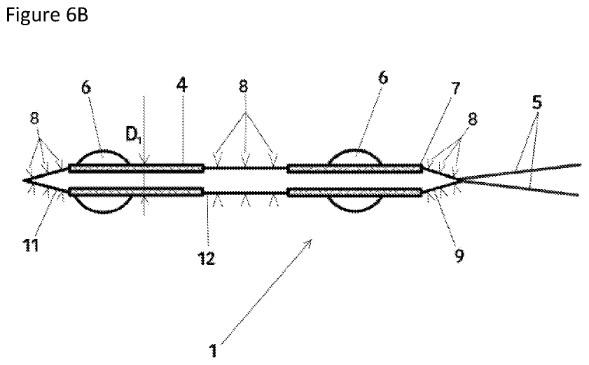

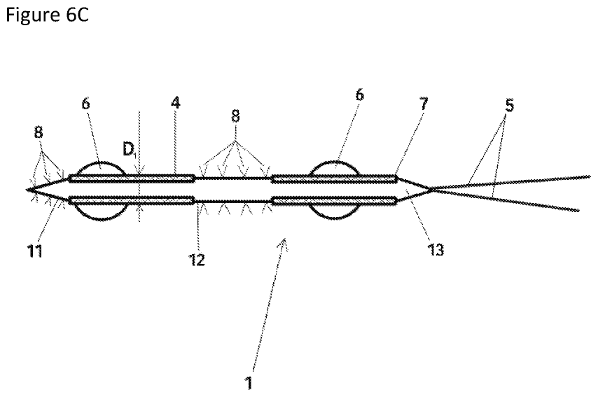

In accordance with another principal aspect of the invention, the device for preventing distal embolism during endovascular interventions includes a first catheter (1) and a second catheter (2). In turn, as illustrated in FIGS. 6A-6C, the first catheter (1) contains a proximal part (5) and a distal part (4). The distal part (4) has a principal through lumen (not shown on the drawings), two lumens (not shown on the drawings) for filling two balloons, two balloons (6) located at a distance from each other, one of which, the distal, is inflated and thus closes the lumen of the right or left common carotid artery, and the other, the proximal, is also inflated and thus closes the left or right subclavian artery, in which case the part (12) of the distal part (4) of the first catheter (1) located between the balloons (6), is made so as to allow blood to flow through said principal lumen but to block the passage of emboli through it. The second catheter (2) from this device is of similar construction to the second catheter (2) of the previously mentioned devices.

The proximal part (5) of the first catheter (1) consists of two lumens for filling two balloons (6) (FIG. 6A-6C). The distal part (4) of the first catheter (1) takes the form of a three-lumen tube comprising: the principal through lumen (not shown in the drawings) the internal diameter of which is sufficient to allow blood to flow through the previously mentioned lumen, and two lumens (not shown in the drawings) used for filling two balloons, narrower than the principal lumen and connected to the previously mentioned two lumens for filling two balloons in the proximal part (5) of the first catheter (1). Each lumen (not shown in the drawings) for filling a balloon in the distal part (4) of the first catheter (1) ends at the place of positioning of the corresponding balloon (6), and the principal through lumen (not shown in the drawings) passes through the place of positioning of the balloons (6) and on to the distal end of the distal part (4) of the first catheter (1).

The balloons (6) in said device, as in the previously described device, may be made of elastomer material.

Also, as mentioned above, the distal part (4) of the first catheter (1) is made of radio-opaque material or contains at least one radio-opaque marker; the principal through lumen of the distal part (4) of the first catheter (1) has an internal diameter of not less than 1 mm but not more than 2.5 mm; the distal part (4) of the first catheter (1) has an external diameter (D₁) of not less than 1.2 mm but not more than 2.8 mm.

In addition, similarly to the previously mentioned devices, the proximal ends of the lumens for filling balloons in the proximal part (5) of the first catheter are closed with an elastic material through which an injection needle can pass.

In turn, in the device according to the second aspect of the invention, the length of the part (12) of the distal part (4) of the first catheter (1), located between the balloons, is 3-4 cm to ensure that the catheter functions in the different anatomical options and vessel lengths.

To ensure that blood can flow through the principal lumen of the distal part (4) of the first catheter (1) and that the passage of emboli through that lumen is simultaneously blocked, the wall of the principal through lumen in the part (12) of the distal part (4) of the first catheter (1) located between the balloons (6) contains holes (8), with the diameter of said holes, made in the wall of the principal through lumen, ranging from 50 to 300 μm and more preferably to 100 μm.

In accordance with another option for the first catheter (1), in order to allow blood to flow through the principal lumen of the distal part (4) of the first catheter (1) and to allow emboli to be blocked simultaneously from flowing through it, the part (12) of the distal part (4) of the first catheter (1) located between the balloons, takes the form of a braided, knitted or meshed polymer anti-embolus filter (made in a similar way to those mentioned above); in this case, the holes in the anti-embolus filter measure from 50 to 300 μm and more preferably to 100 μm.

In accordance with the option for the first catheter shown in FIG. 6A, the proximal end (7) of the principal through lumen of the distal part (4) of the first catheter (1) is made completely open.

In accordance with the alternative option for the first catheter shown in FIG. 6B, the proximal part (7) of the principal through lumen of the distal part (4) of the first catheter (1) contains an anti-embolus filter (9), taking the form of a braided, knitted or meshed conical polymer filter, the length of which is within the range 2 cm to 40 cm, preferably to 30 cm and more preferably to 20 cm, in which case the wholes (8) in the anti-embolus filter (9) may vary from 50 to 300 μm and more preferably to 100 μm.

Additionally, said anti-embolus filter (9) contains a hole (not shown in the drawing), the diameter of which is equal to that of the guidewire passed through when the first catheter (1) is inserted into the vessel through said hole into the principal through lumen of the distal part (4) of the first catheter (1).

In accordance with the option for the first catheter shown in FIG. 6C, the proximal end (7) of the principal through lumen of the distal part of the first catheter (1) is made closed (13) with a hole equal in diameter to that of the guidewire passed through when the first catheter (1) is inserted into the vessel through said hole into the principal through lumen of the distal part (4) of the first catheter (1).

All of the above options for the first catheter, without being limited to the illustrations in FIGS. 6B-6C, may, in order to provider better prevention of embolism, contain an additional anti-embolus filter (11) which closes the distal end of the principal through lumen of the distal part (4) of the first catheter (1).

And finally, as an additional element to the first and second catheter, the device for preventing distal embolism during endovascular interventions, in accordance with this aspect of the invention, may also contain a third catheter (3) (FIG. 2), designed to introduce a liquid medicinal preparation and made in the form of a single-lumen tube with a proximal end and a distal end.

In this case, the proximal and distal parts (5, 4) of the first catheter (1) have external diameters sufficient to allow passage through the lumen of the third catheter (3), and combined lengths sufficient to ensure that at least the distal part of the first catheter (1) extends beyond the limits of the distal end of the third catheter (3), when the first catheter (1) passes through the third catheter (3)1 in addition, the internal diameter (D₃) of the lumen of the third catheter (3) is equal to the external diameter (D₁) of the distal part of the first catheter (1): D₃=D₁.

The above-mentioned options for the kit are used as follows:

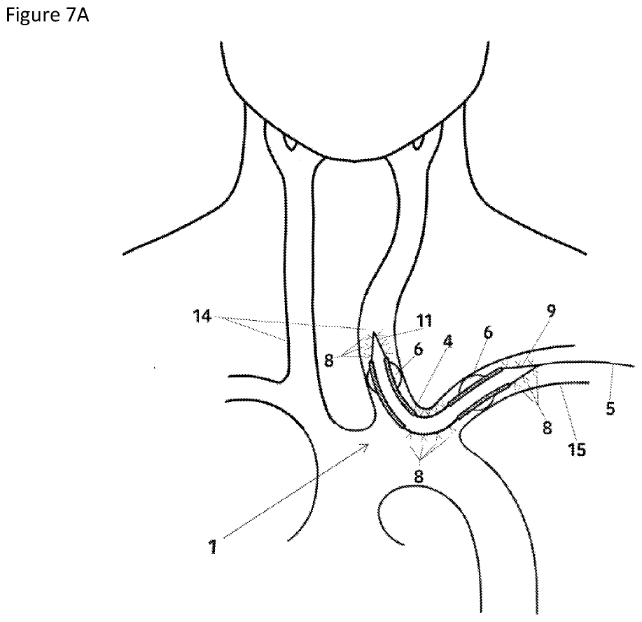

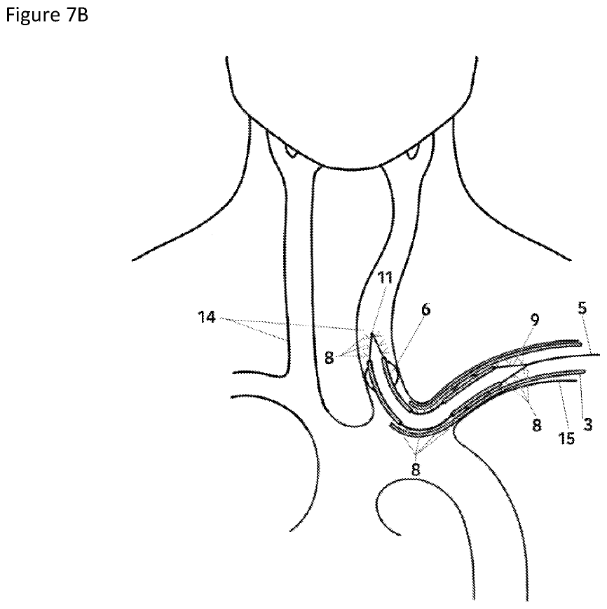

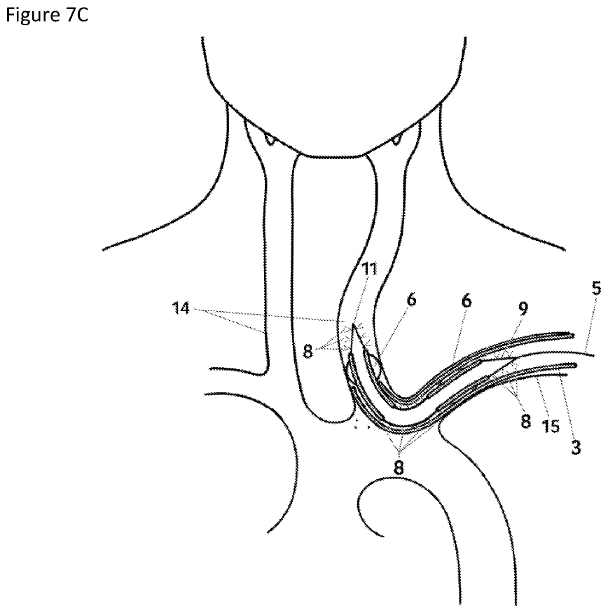

Using the generally known method, the radial artery or the brachial artery is injected. The introduced is then inserted into said radial artery or brachial artery respectively. Through said introducer, the guidewire is then inserted into the left or right common carotid artery (14). The first catheter (1) is inserted through the introducer using said guidewire so that the distal balloon (6) is placed in the left or right common carotid artery (14) respectively and the proximal balloon (6) is placed in the left or right subclavian artery (15). First, through one lumen for filling a balloon (6), the distal balloon of said first catheter (1) is inflated until the distal balloon closes the lumen of the previously mentioned left or right common carotid artery (14), and then through the other lumen for filling a balloon (6) the proximal balloon (6) of said first catheter (1) is inflated until the proximal balloon (6) closes the lumen of the previously mentioned left or right subclavian artery (15) (FIG. 7A). A third catheter (3) is inserted through the introducer into said first catheter (1) from the proximal end; then the proximal balloon (6) of the previously mentioned first catheter (1) is inflated; then the previously mentioned third catheter (3) is moved all the way to the distal balloon (6) of said first catheter (1) (FIG. 7B). Then, the liquid medical preparation (similar to the above-mentioned) is introduced into the third catheter (FIG. 7B). After all the medicinal preparation has been introduced, the third catheter (3) is removed. The proximal balloon (6) of the previously mentioned first catheter (1) is then inflated until the proximal balloon (6) closes the previously mentioned left or right subclavian artery (15). After the endovascular intervention has been completed in the ascending thoracic aorta and the thoracic aortic arch, a second catheter (2) is applied to said first catheter (1), the proximal balloon (6) of the previously mentioned first catheter (1) is deflated, the previously mentioned second catheter (2) is brought to the inflated distal balloon (6) of the previously mentioned first catheter (1) and the a liquid substance, such as saline, is swiftly introduced into said second catheter (2); any emboli remaining in the spaces around the distal balloon (6) are flushed into the distal blood flow in the previously mentioned first catheter in the left or right common carotid artery (14) (FIG. 7C). Said second catheter (2) is then removed, the distal balloon (6) of the previously mentioned first catheter (1) is deflated, and the previously mentioned first catheter (1), guidewire and introducer are removed.

It should also be noted that when the first catheter (1) is used with the proximal end (7) of its distal part (4) closed (13) (FIG. 6C), or when there is no requirement to inject medications into the distal area of the endovascular intervention, a device consisting only of a first and second catheter is used, and accordingly, such steps of the method as applying the third catheter to the first catheter, introducing a medical preparation and removing the third catheter, are not carried out.

In addition, it should be noted that the above-mentioned devices, as well as those mentioned earlier, may be used to prevent embolism in cerebral arteries during endovascular aortic valve replacement, and in endoprosthetics of the ascending and thoracic aortic arch; it is obvious to the specialist that for more effective protection against embolism, several devices simultaneously, for example, two, may be used, and if the distal balloon of the first catheter from the first set is inserted in either the right or the left common carotid artery, then the distal balloon of the first catheter from the other (second) set, respectively, is inserted in the other left or right common carotid artery.

Representative embodiments of the invention are as follows:
1. A kit for prevention of distal embolism during endovascular interventions, comprising:
   a first catheter, comprising:
      a proximal part and a distal part, having a principal through lumen,
      at least one lumen for filling, at least one balloon,
      and at least one inflated balloon, which closes the lumen of the right or left common carotid artery, and
      a proximal end, made so as to allow a flow of blood through the above-mentioned principal lumen but also to block the passage of emboli through it; and
   a second catheter, made in the form of a single-lumen tube, with a proximal end and distal end;
      in which the proximal and distal parts of the first catheter have external diameters of sufficient dimensions to allow passage through the lumen of the second catheter, and are of sufficient length to allow at least the distal part of the first catheter to extend beyond the limits of the distal end of the second catheter when the first catheter passes through the second catheter;
      in which case the internal diameter of the lumen of the second catheter exceeds the external diameter of the distal part of the first catheter.
2. A kit according to embodiment 1, in which
   the proximal part of the first catheter consists of at least one lumen for filling and at least one balloon; and
   the distal part of the first catheter takes the form of a tube with at least two lumens, this tube comprising:
      a principal through lumen of sufficient internal diameter to allow a flow of blood through the above-mentioned principal lumen, and
      at least one lumen for filling, at least one balloon that is narrower than the principal through lumen and connects with the one previously mentioned, at least one lumen for filling at least one balloon in the proximal part of the first catheter.
3. A kit according to either of embodiments 1 or 2, in which at least one lumen for filling at least one balloon in the distal part of the first catheter ends at the place of positioning of at least one balloon, and the principal through lumen passes through the place of positioning of at least one balloon and as far as the distal end of the distal part of the first catheter.
4. A kit according to any one of embodiments 1-3, in which at least one balloon is made of an elastomer material.
5. A kit according to any one of embodiments 1-4, in which the distal part of the first catheter is made of radio-opaque material or contains at least one radio-opaque marker.
6. A kit according to any one of embodiments 1-5, in which the distal part of the first catheter has a
   principal through lumen,
   one lumen for filling one balloon and
   one inflated balloon, which closes the lumen of the right or left common carotid artery and is positioned on the side of the distal end in the above-mentioned distal part, and the
   proximal part of the first catheter consists of one lumen for filling one balloon.
7. A kit according to embodiment 6, within which an anti-embolus filter us positioned on the proximal end of the principal through lumen of the first catheter.
8. A kit according to embodiment 7, within which the anti-embolus filter is a set of through holes made in the wall of the principal through lumen at the proximal end of the principal through lumen of the distal part of the first catheter.
9. A kit according to embodiment 8, in which the principal through lumen of the distal part of the first catheter is cylindrical throughout its length.
10. A kit according to embodiment 9, in which the above-mentioned holes made in the wall of the principal through lumen are located at a distance of at least 20 cm and not more than 70 cm from the balloon and measure from 50 to 300 μm in diameter and more preferably up to 100 μm.
11. A kit according to embodiment 8, in which the principal through lumen of the distal part of the first catheter narrows conically at its proximal end.
12. A kit according to embodiment 11, in which the length of the conically narrowing proximal end of the principal through lumen of the distal part of the first catheter is within a range of 2 cm to 40 cm, preferably to 30 cm and more preferably to 20 cm.
13. A kit according to either of embodiments 11 or 12, in which the above-mentioned holes made in the wall of the principal through lumen are 50 to 300 μm in diameter and preferably to 100 μm.
14. A kit according to embodiment 7, in which the anti-embolus filter is a braided, knitted or meshed polymer conical filter.
15. A kit according to embodiment 14, in which the length of the conical filter is within a range of 2 to 40 cm, preferably to 30 cm and more preferably to 20 cm.
16. A kit according to embodiment 7, in which the anti-embolus filter is a network that closes the proximal hole in the principal through lumen.
17. A kit according to embodiment 7, in which the anti-embolus filter is a porous membrane that closes the proximal hole of the principal through lumen.
18. A kit according to any one of embodiments 1-5, in which the distal part of the first catheter has a principal through lumen,
   two lumens for filling balloons and
   two inflated balloons located at a certain distance from each other which close the lumen of the right or left common carotid artery, and the
   proximal part of the first catheter consists of two lumens for filling two balloons.
19. A kit according to embodiment 18, in which an anti-embolus filter is provided at the proximal end of the principal through lumen of the first catheter.
20. A kit according to embodiment 19, in which the anti-embolus filter is a braided, knitted or meshed polymer conical filter.
21. A kit according to embodiment 19, in which the anti-embolus filter is a network that closes the proximal hole in the principal through lumen.
22. A kit according to embodiment 19, in which the anti-embolus filter is a porous membrane that closes the proximal hole of the principal through lumen.
23. A kit according to any one of embodiments 18-22, within which the distance between the balloons is 3-4 cm.
24. A kit according to any one of embodiments 1-8 and 11-23, in which the end-point of the proximal end of the principal through lumen of the distal part of the first catheter contains a hole equal in diameter to the channel that passes through said opening in the principal through lumen of the distal part of the first catheter.
25. A kit according to any one of embodiments 1-24, in which the principal through lumen of the distal part of the first catheter has an internal diameter of not less than 1 mm, but not more than 2.5 mm.
26. A kit according to any one of embodiments 1-25, in which the distal part of the first catheter has an external diameter of not less than 1.2 mm but not more than 2.8 mm.
27. A kit according to any one of embodiments 1-26, in which the proximal end of at least one lumen for filling and at least one balloon in the proximal part of the first catheter is closed with an elastic material through which an injection needle can be passed.
28. A kit according to any one of embodiments 1-27, in which the distal end of the principal through lumen of the distal part of the first catheter is closed with an additional anti-embolus filter.
29. A kit according to any one of embodiments 1-28, additionally comprising:
   a third catheter in the form of a single-lumen tube with a proximal end and a distal end;
      in which case the proximal and distal parts of the first catheter have external diameters sufficient to allow passage through the lumen of the third category and lengths which together are sufficient to allow at least the distal part of the first catheter to extend beyond the limits of the distal end of the third catheter, when the first catheter passes through the third catheter;
      in which case the internal diameter of the lumen of the third catheter is essentially the same as the external diameter of the distal part of the first catheter.
30. A kit for prevention of distal embolism during endovascular interventions, comprising:
   a first catheter, comprising:
      a proximal part and a
      distal part, having a
         principal through lumen that is cylindrical throughout its length,
         a lumen for filling a balloon,
         an inflated balloon that closes the lumen of the right or left common carotid artery and is positioned on the above-mentioned distal part from its distal end, and an
         anti-embolus filter provided at the proximal end of the principal through lumen of the distal part of the first catheter; and
   a second catheter, made in the form of a single-lumen tube, with a proximal end and distal end;
      in which the proximal and distal parts of the first catheter have external diameters of sufficient dimensions to allow passage through the lumen of the second catheter, and are of sufficient length to allow at least the distal part of the first catheter to extend beyond the limits of the distal end of the second catheter when the first catheter passes through the second catheter;
      in this case the internal diameter of the lumen of the second catheter exceeds the external diameter of the distal part of the first catheter;
      in this case, the anti-embolus filter is a series of through holes made in the wall of the principal through lumen on the proximal end of the principal through lumen of the distal part of the first catheter and positioned not less than 20 cm and not more than 70 cm from the balloon and having a diameter of not less than 50 μm and not more than 300 μm.
31. A kit according to embodiment 30, in which
   the proximal part of the first catheter consists of one lumen for filling the balloon; and
   the distal part of the first catheter is a two-lumen tube that includes:
      a principal through lumen of sufficient internal diameter to allow a flow of blood through the above-mentioned principal lumen, and
      a lumen, for filling the balloon, which is narrower than the principal through lumen and is connected to the above-mentioned lumen for filling the balloon with the proximal part of the first lumen.
32. A kit according to either of embodiments 30 or 31, in which the lumen for filling the balloon with the distal part of the first catheter ends at the position of the balloon and the principal through lumen passes through the position of the balloon and as far as the distal end of the distal part of the first catheter.
33. A kit according to any one of embodiments 30-32, in which the balloon is made of an elastomer.
34. A kit according to any one of embodiments 30-33, in which the distal part of the first catheter is made of a radio-opaque material or contains at least one radio-opaque marker.
35. A kit according to any one of embodiments 30-34, in which the principal through lumen of the distal part of the first catheter has an internal diameter of not less than 1 mm but not more than 2.5 mm.
36. A kit according to any one of embodiments 30-35, in which the distal part of the first catheter has an external diameter of not less than 1.2 mm but not more than 2.8 mm.
37. A kit according to any one of embodiments 30-36, in which the proximal end of the lumen for filling the balloon in the proximal part of the first catheter is closed with an elastic material through which an injection needle can be passed.
38. A kit according to any one of embodiments 30-37, in which the distal end of the principal through lumen of the distal part of the first catheter is closed using an additional anti-embolus filter.
39. A kit according to any one of embodiments 30-38, additionally comprising:
   a third catheter in the form of a single-lumen tube with a proximal end and a distal end;
      in which case the proximal and distal parts of the first catheter have external diameters sufficient to allow passage through the lumen of the third category and lengths which together are sufficient to allow at least the distal part of the first catheter to extend beyond the limits of the distal end of the third catheter, when the first catheter passes through the third catheter;
      in which case the internal diameter of the lumen of the third catheter is essentially the same as the external diameter of the distal part of the first catheter.
40. A kit for prevention of distal embolism during endovascular interventions, comprising:
   a first catheter, comprising:
      a proximal part and a
      distal part, having a
         principal through lumen, conically narrowing at its proximal end,
         a lumen for filling a balloon,
         an inflated balloon that closes the lumen of the right or left common carotid artery and is positioned on the above-mentioned distal part from its distal end, and an
         anti-embolus filter provided at the proximal end of the principal through lumen of the distal part of the first catheter; and
   a second catheter in the form of a single-lumen tube with
      a proximal end and a distal end,
      in which the proximal and distal parts of the first catheter have external diameters of sufficient dimensions to allow passage through the lumen of the second catheter, and are of sufficient length to allow at least the distal part of the first catheter to extend beyond the limits of the distal end of the second catheter when the first catheter passes through the second catheter;
      in this case the internal diameter of the lumen of the second catheter exceeds the external diameter of the distal part of the first catheter;
      in which case, the anti-embolus filter is a series of through holes made in the wall of the principal through lumen at the proximal end of the principal through lumen of the distal part of the first catheter;
      in this case the length of the conically narrowing proximal end of the principal through lumen of the distal part of the first catheter is within the range of 2 cm to 40 cm, and the above-mentioned holes made in the wall of the principal through lumen are of a diameter not less than 50 μm and not more than 300 μm;
      in this case, the end section of the proximal end of the principal through lumen of the distal part of the first catheter contains a hole of a diameter equal to the diameter of the guidewire that passes through said hole into the principal through lumen of the distal part of the first catheter.
41. A kit according to embodiment 40, in which
   the proximal part of the first catheter consists of one lumen for filling the balloon; and
   the distal part of the first catheter is a two-lumen tube that includes:
      a principal through lumen of sufficient internal diameter to allow a flow of blood through the above-mentioned principal lumen, and
      a lumen, for filling the balloon, which is narrower than the principal through lumen and is connected to the above-mentioned lumen for filling the balloon with the proximal part of the first lumen.
42. A kit according to either of embodiments 40 or 41, in which the lumen for filling the balloon in the distal part of the first catheter ends at the place of positioning of the balloon, and the principal through lumen passes through the place of positioning of the balloon as far as the distal end of the distal part of the first catheter.
43. A kit according to any one of embodiments 40-42, in which the balloon is made of an elastomer material.
44. A kit according to any one of embodiments 40-43, in which the distal part of the first catheter is made of radio-opaque material or contains at least one radio-opaque marker.
45. A kit according to any one of embodiments 40-44, in which the principal through lumen of the distal part of the first catheter has an internal diameter of not less than 1 mm but not more than 2.5 mm.
46. A kit according to any one of embodiments 40-45, in which the distal part of the first catheter has an external diameter of not less than 1.2 mm but not more than 2.8 mm.
47. A kit according to any one of embodiments 40-46, in which the proximal end of the lumen for filling the balloon in the proximal part of the first catheter is closed with an elastic material through which an injection needle can be passed.
48. A kit according to any one of embodiments 40-47, in which the length of the conically narrowing proximal part of the principal through lumen of the distal part of the first catheter is within a range of 2 cm to 30 cm and preferably to 20 cm.
49. A kit according to any one of embodiments 40-48, in which the distal end of the principal through lumen of the distal part of the first catheter is closed with an additional anti-embolus filter.
50. A kit according to any one of embodiments 40-49, additionally comprising a
   third catheter, taking the form of a single lumen tube having
   a proximal end and a distal end,
      in which case the proximal and distal parts of the first catheter have external diameters sufficient to allow passage through the lumen of the third category and lengths which together are sufficient to allow at least the distal part of the first catheter to extend beyond the limits of the distal end of the third catheter, when the first catheter passes through the third catheter;
      in which case the internal diameter of the lumen of the third catheter is essentially the same as the external diameter of the distal part of the first catheter.
51. A kit for prevention of distal embolism during endovascular interventions, comprising:
   a first catheter, comprising:
      a proximal part and a
      distal part, having a
         principal through lumen,
         a lumen for filling a balloon,
         an inflated balloon that closes the lumen of the right or left common carotid artery and is positioned on the above-mentioned distal part from its distal end, and an
         anti-embolus filter provided at the proximal end of the principal through lumen of the distal part of the first catheter; and
   a second catheter in the form of a single-lumen tube with
      a proximal end and a distal end,
      in which the proximal and distal parts of the first catheter have external diameters of sufficient dimensions to allow passage through the lumen of the second catheter, and are of sufficient length to allow at least the distal part of the first catheter to extend beyond the limits of the distal end of the second catheter when the first catheter passes through the second catheter;
      in this case the internal diameter of the lumen of the second catheter exceeds the external diameter of the distal part of the first catheter;
      in this case the anti-embolus filter is a braided, knitted or meshed polymer conical filter of a length within the range 2 cm to 40 cm.
52. A kit according to embodiment 51, in which
   the proximal part of the first catheter consists of one lumen for filling the balloon; and
   the distal part of the first catheter is a two-lumen tube that includes:
      a principal through lumen of sufficient internal diameter to allow a flow of blood through the above-mentioned principal lumen, and
      a lumen, for filling the balloon, which is narrower than the principal through lumen and is connected to the above-mentioned lumen for filling the balloon with the proximal part of the first lumen.
53. A kit according to any one of embodiments 51-52, in which the lumen for filling the balloon in the distal part of the first catheter ends at the place of positioning of the balloon, and the principal through lumen passes through the place of positioning of the balloon and to the distal end of the distal part of the first catheter.
54. A kit according to any one of embodiments 51-53, in which the balloon is made of an elastomer material.
55. A kit according to any one of embodiments 51-54, in which the distal part of the first catheter is made of radio-opaque material or contains at least one radio-opaque marker.
56. A kit according to any one of embodiments 51-55, in which the principal through lumen of the distal part of the first catheter has an internal diameter of not less than 1 mm, but not more than 2.5 mm.
57. A kit according to any one of embodiments 51-56, in which the distal part of the first catheter has an external diameter of not less than 1.2 mm, but not more than 2.8 mm.
58. A kit according to any one of embodiments 51-57, in which the proximal end of the lumen for filling the balloon of the proximal part of the first catheter is closed with an elastic material through which an injection needle can be passed.
59. A kit according to any one of embodiments 51-58, in which the length of the anti-embolus filter is within a range of 2 cm to 30 cm, preferably to 20 cm.
60. A kit according to any one of embodiments 51-59, in which the holes in the anti-embolus filter are 50 to 300 μm in diameter and more preferably to 100 μm.
61. A kit according to any one of embodiments 51-60, in which the anti-embolus filter contains an additional hole equal in diameter to the guidewire passing through said hole into the principal through lumen of the distal part of the first catheter.
62. A kit according to any one of embodiments 51-61, in which the distal end of the principal through lumen of the distal party of the first catheter is closed with an additional anti-embolus filter.
63. A kit according to any one of embodiments 51-62, additionally comprising:
   a third catheter in the form of a single-lumen tube with a proximal end and a distal end;
      in which case the proximal and distal parts of the first catheter have external diameters sufficient to allow passage through the lumen of the third category and lengths which together are sufficient to allow at least the distal part of the first catheter to extend beyond the limits of the distal end of the third catheter, when the first catheter passes through the third catheter;
      in which case the internal diameter of the lumen of the third catheter is essentially the same as the external diameter of the distal part of the first catheter.
64. A kit for prevention of distal embolism during endovascular interventions, comprising:
   a first catheter, comprising:
      a proximal part and a
      distal part, having a
         principal through lumen,
         a lumen for filling a balloon,
         an inflated balloon that closes the lumen of the right or left common carotid artery and is positioned on the above-mentioned distal part from its distal end, and an
         anti-embolus filter provided at the proximal end of the principal through lumen of the distal part of the first catheter; and
   a second catheter in the form of a single-lumen tube with
      a proximal end and a distal end,
      in which the proximal and distal parts of the first catheter have external diameters of sufficient dimensions to allow passage through the lumen of the second catheter, and are of sufficient length to allow at least the distal part of the first catheter to extend beyond the limits of the distal end of the second catheter when the first catheter passes through the second catheter;
      in this case the internal diameter of the lumen of the second catheter exceeds the external diameter of the distal part of the first catheter;
      in this case the anti-embolus filter takes the form of a network that closes the proximal hole in the principal through lumen.
65. A kit according to embodiment 64, in which
   the proximal part of the first catheter consists of one lumen for filling the balloon; and
   the distal part of the first catheter is a two-lumen tube that includes:
      a principal through lumen of sufficient internal diameter to allow a flow of blood through the above-mentioned principal lumen, and
      a lumen, for filling the balloon, which is narrower than the principal through lumen and is connected to the above-mentioned lumen for filling the balloon with the proximal part of the first lumen.
66. A kit according to any one of embodiments 64-65, in which the lumen for filling the balloon of the distal part of the first catheter ends at the place of positioning of the balloon, and the principal through lumen passes through the place of positioning of the balloon and as far as the distal end of the distal part of the first catheter.
67. A kit according to any one of embodiments 64-66, in which the balloon is made of an elastomer material.
68. A kit according to any one of embodiments 64-67, in which the distal part of the first catheter is made of radio-opaque material or contains at least one radio-opaque marker.
69. A kit according to any one of embodiments 64-68, in which the principal through lumen of the distal part of the first catheter has an internal diameter of not less than 1 mm but not more than 2.5 mm.
70. A kit according to any one of embodiments 64-69, in which the distal part of the first catheter has an external diameter of not less than 1.2 mm, but not more than 2.8 mm.
71. A kit according to any one of embodiments 64-70, in which the proximal part of the lumen for filling the balloon in the proximal part of the first catheter is closed with an elastic material through which an injection needle can be passed.
72. A kit according to any one of embodiments 64-71, in which the angle α of inclination of the above-mentioned network covering the proximal hole in the principal through lumen is within a range of 0°<α≤90°.
73. A kit according to any one of embodiments 64-72, in which the holes in the above-mentioned network covering the proximal hole in the principal through lumen measures from 50 to 300 μm and more preferably to 100 μm.
74. A kit according to any one of embodiments 64-73, in which the above-mentioned network contains an additional hole the diameter of which equals the diameter of the guidewire passing through said hole into the principal through lumen of the distal part of the first catheter.
75. A kit according to any one of embodiments 64-74, in which the distal end of the principal through lumen of the distal part of the first catheter is covered with an additional anti-embolus filter.
76. A kit according to any one of embodiments 64-75, additionally comprising:
   a third catheter in the form of a single-lumen tube with a proximal end and a distal end;
      in which case the proximal and distal parts of the first catheter have external diameters sufficient to allow passage through the lumen of the third category and lengths which together are sufficient to allow at least the distal part of the first catheter to extend beyond the limits of the distal end of the third catheter, when the first catheter passes through the third catheter;
      in which case the internal diameter of the lumen of the third catheter is essentially the same as the external diameter of the distal part of the first catheter.
77. A kit for prevention of distal embolism during endovascular interventions, comprising:
   a first catheter, comprising:
      a proximal part and a
      distal part, having a
         principal through lumen,
         two lumens for filling two balloons,
         two balloons located at a distance from each other and inflated, thus covering the lumen of the right or left common carotid artery, and
         anti-embolus filter provided at the proximal end of the principal through lumen of the distal part of the first catheter; and
   a second catheter, made in the form of a single-lumen tube, with a proximal end and distal end;
      in which the proximal and distal parts of the first catheter have external diameters of sufficient dimensions to allow passage through the lumen of the second catheter, and are of sufficient length to allow at least the distal part of the first catheter to extend beyond the limits of the distal end of the second catheter when the first catheter passes through the second catheter;
      in this case the diameter of the lumen of the second catheter exceeds the external diameter of the distal part of the first catheter;
      in this case the anti-embolus filter is a braided, knitted or meshed polymer conical filter of a length within the range 2 cm to 40 cm.
78. A kit according to embodiment 77, in which
   the proximal part of the first catheter consists of two lumens for filling two balloons; and
   the distal part of the first catheter takes the form of a three-lumen tube, comprising:
      a principal through lumen of sufficient internal diameter to allow a flow of blood through the above-mentioned principal lumen, and
      two lumens for filling two balloons, which are narrower than the principal through lumen and connect with the previously mentioned two lumens for filling the two balloons in the proximal part of the first catheter.
79. A kit according to either of embodiments 77 or 78, in which each lumen for filling the balloon in the distal part of the first catheter ends at the place of positioning of the corresponding balloon, and the principal through lumen passes through the place of positioning of the balloons as far as the distal end of the distal part of the first catheter.
80. A kit according to any one of embodiments 77-79, in which the balloons are made of an elastomer material.
81. A kit according to any one of embodiments 77-80, in which the distal part of the first catheter is made of radio-opaque material or contains at least one radio-opaque marker.
82. A kit according to any one of embodiments 77-81, in which the principal through lumen of the distal part of the first catheter has an internal diameter of not more than 1 mm but not more than 2.5 mm.
83. A kit according to any one of embodiments 77-82, in which the distal part of the first catheter has an external diameter of not less than 1.2 mm, but not more than 2.8 mm.
84. A kit according to any one of embodiments 77-83, in which the proximal ends of the lumens for filling the balloons in the proximal part of the first catheter are closed with an elastic material through which an injection needle can be passed.
85. A kit according to any one of embodiments 77-84, in which the anti-embolus filter is within a range of 2 cm to 30 cm, preferably to 20 cm.
86. A kit according to any one of embodiments 77-85, in which the holes in the anti-embolus filter are 50 to 300 μm in diameter and more preferably to 100 μm.
87. A kit according to any one of embodiments 77-86, in which the anti-embolus filter contains an additional hole equal in diameter to the guidewire passing through said hole into the principal through lumen of the distal part of the first catheter.
88. A kit according to any one of embodiments 77-87, in which the distance between the balloons is 3-4 cm.
89. A kit according to any one of embodiments 77-88, in which the distal end of the principal through lumen of the distal part of the first catheter is closed with an additional anti-embolus filter.
90. A kit according to any one of embodiments 77-89, additionally comprising:
   a third catheter in the form of a single-lumen tube with a proximal end and a distal end;
      in which case the proximal and distal parts of the first catheter have external diameters sufficient to allow passage through the lumen of the third category and lengths which together are sufficient to allow at least the distal part of the first catheter to extend beyond the limits of the distal end of the third catheter, when the first catheter passes through the third catheter;
      in which case the internal diameter of the lumen of the third catheter is essentially the same as the external diameter of the distal part of the first catheter.
91. A method of preventing distal embolism during endovascular interventions using the kit according to any one of embodiments 1-28, 30-38, 40-49, 51-62, 64-75 and 77-89, including steps of:
   injecting the common femoral artery, the radial artery or the brachial artery;
   inserting the introducer into said common femoral artery, radial artery or brachial artery;
   inserting the guidewire through the introducer into the left or right common carotid artery;
   inserting the first catheter through the introducer using said guidewire;
   inflating, through at least one lumen for filling at least one balloon in the first catheter, at least one balloon in said first catheter, until the lumen of said right or left common carotid artery is closed;
   placing, after the endovascular intervention is completed in the ascending thoracic aorta and the aortic arch, the second catheter on the first catheter;
   bringing said second catheter up to the inflated balloon in the first catheter and introducing a liquid substance swiftly into the second catheter to allow flushing of any emboli remaining in the areas around the balloon into the distal blood flow behind the aortic arch before the first catheter in the left or right common carotid artery;
   removing the second catheter;
   inflating at least one balloon in the first catheter;
   removing the first catheter, guidewire and introducer, as mentioned above.
92. Method according to embodiment 91, in which a kit including the first catheter, comprising one balloon and one lumen for filling the balloon, is used.
93. Method according to embodiment 91, in which a kit including the first catheter, comprising two balloons and two lumens for filling the balloons, are used.
94. Method according to any one of embodiments 91-93, where the liquid used for flushing out the emboli is saline solution.
95. Method according to any one of embodiments 91-94, in which the endovascular procedure is an endovascular aortic valve replacement.
96. Method of preventing distal embolism during endovascular interventions using a kit according to any one of embodiments 29, 39, 50, 63, 76 and 90, including steps of:
   injecting the common femoral artery, the radial artery or the brachial artery is injected;
   inserting the introducer into said common femoral artery, radial artery or brachial artery;
   inserting the guidewire through the introducer into the left or right common carotid artery;
   inserting the first catheter using the guidewire through the above-mentioned introducer;
   inflating, through at least one lumen for filling at least one balloon in the first catheter, at least one balloon in said first catheter, until the lumen of said right or left common carotid artery is closed;
   inserting a third catheter through the introducer into the first catheter from the proximal end until the balloon in the first catheter is reached;
   introducing the liquid medicinal preparation into the third catheter;
   removing the third catheter;
   placing, after the endovascular intervention is completed in the ascending thoracic aorta and the aortic arch, the second catheter on the first catheter;
   bringing said second catheter up to the inflated balloon in the first catheter and introducing a liquid substance swiftly into the second catheter to allow flushing of any emboli remaining in the areas around the balloon into the distal blood flow behind the aortic arch before the first catheter in the left or right common carotid artery;
   removing the second catheter;
   inflating at least one balloon in the first catheter;
   removing the first catheter, guidewire and introducer, as mentioned above.
97. Method according to embodiment 96, where a kit including a first catheter comprising one balloon and one lumen for filling the balloon is used.
98. Method according to embodiment 96, where a kit including a first catheter comprising two balloons and two lumens for filling the balloons are used.
99. Method according to any one of embodiments 96-98, where the liquid used to flush out the emboli is saline solution.
100. Method according to any one of embodiments 96-99, where the endovascular intervention is an endovascular aortic valve replacement.
101. A kit for prevention of distal embolism during endovascular interventions, comprising:
   a first catheter, comprising:
      a proximal part and a
      distal part, having a
         principal through lumen,
         two lumens for filling two balloons,
         two balloons placed at a distance from each other, one of which, the distal, is inflated and thus closes the lumen of the right or left common carotid artery, and the other, the proximal, is inflated and thus closes the lumen of the left or right subclavian artery; and
   a second catheter, made in the form of a single-lumen tube, with a proximal end and distal end;
      in which the proximal and distal parts of the first catheter have external diameters of sufficient dimensions to allow passage through the lumen of the second catheter, and are of sufficient length to allow at least the distal part of the first catheter to extend beyond the limits of the distal end of the second catheter when the first catheter passes through the second catheter;
      in this case the internal diameter of the lumen of the second catheter exceeds the external diameter of the distal part of the first catheter;
      in this case, the part of the distal part of the catheter located between the balloons is made so as to allow blood to flow through said principal lumen but to block the passage of emboli through it.
102. A kit according to embodiment 101, in which
   the proximal part of the first catheter consists of two lumens for filling two balloons; and
   the distal part of the first catheter takes the form of a three-lumen tube, comprising:
      a principal through lumen of sufficient internal diameter to allow a flow of blood through the above-mentioned principal lumen, and
      two lumens for filling two balloons, which are narrower than the principal through lumen and connect with the previously mentioned two lumens for filling the two balloons in the proximal part of the first catheter.
103. A kit according to either of embodiments 101 or 102, in which each lumen for filling the balloons in the distal part of the first catheter ends at the place of positioning of the corresponding balloon, and the principal through lumen passes through the place of positioning of the balloons as far as the distal end of the distal part of the first catheter.
104. A kit according to any one of embodiments 101-103, in which the balloons are made of an elastomer material.
105. A kit according to any one of embodiments 101-104, in which the distal part of the first catheter is made of radio-opaque material or contains at least one radio-opaque marker.
106. A kit according to any one of embodiments 101-105, in which the principal through lumen of the distal part of the first catheter has an internal diameter of not less than 1 mm but not more than 2.5 mm.
107. A kit according to any one of embodiments 101-106, in which the distal part of the first catheter has an external diameter of not less than 1.2 mm but not more than 2.8 mm.
108. A kit according to any one of embodiments 101-107, in which the proximal ends of the lumens for filling the balloons in the proximal part of the first catheter are closed using an elastic material through which an injection needle can be passed.
109. A kit according to any one of embodiments 101-108, in which the length of the part of the distal part of the first catheter, located between the balloons, is 3-4 cm.
110. A kit according to any one of embodiments 101-109, in which the wall of the principal through lumen of the part of the distal part of the first catheter, located between the balloons, contains holes.
111. A kit according to embodiment 110, in which said holes made in the wall of the principal through lumen are from 50 to 300 μm in diameter, more preferably 100 μm.
112. A kit according to any one of embodiments 101-109, in which the part of the distal part of the first catheter located between the balloons takes the form of a braided, knitted or meshed anti-embolus filter.
113. A kit according to embodiment 112, in which the holes in the anti-embolus filter are from 50 to 300 mm in diameter, more preferably to 100 μm.
114. A kit according to any one of embodiments 101-113, in which the proximal end of the principal through lumen of the distal part of the first catheter is completely open.
115. A kit according to any one of embodiments 101-113, in which an anti-embolus filter is located in the proximal end of the principal through lumen of the distal part of the first catheter.
116. A kit according to embodiment 115, in which the anti-embolus filter positioned in the proximal end of the principal through lumen of the distal part of the first catheter takes the form of a braided, knitted of meshed conical polymer filter, the length of which ranges from 2 cm to 40 cm, preferably to 30 cm and more preferably to 20 cm.
117. A kit according to either of embodiments 115 or 116, in which the holes in the anti-embolus filter positioned at the proximal end of the principal through lumen of the distal part of the first catheter measure from 50 to 300 μm, more preferably to 100 μm.
118. A kit according to any one of embodiments 115-117, in which the anti-embolus filter positioned at the proximal end of the principal through lumen of the distal part of the first catheter contains an additional hole equal in diameter to the guidewire passing through said hole into the principal through lumen of the distal part of the first catheter.
119. A kit according to any one of embodiments 101-113, in which the proximal end of the principal through lumen of the distal part of the first catheter is closed, with a hole equal in diameter to that of the guidewire passing through said hole into the principal through lumen of the distal part of the first catheter.
120. A kit according to any one of embodiments 101-118, in which the distal end of the principal through lumen of the distal part of the first catheter is closed with an additional anti-embolus filter.
121. A kit according to embodiment 119, in which the distal end of the principal through lumen of the distal part of the first catheter is closed with an additional anti-embolus filter.
122. A kit according to any one of embodiments 101-118 and 120, additionally comprising:
   a third catheter in the form of a single-lumen tube with a proximal end and a distal end;
      in which case the proximal and distal parts of the first catheter have external diameters sufficient to allow passage through the lumen of the third category and lengths which together are sufficient to allow at least the distal part of the first catheter to extend beyond the limits of the distal end of the third catheter, when the first catheter passes through the third catheter;
      in which case the internal diameter of the lumen of the third catheter is essentially the same as the external diameter of the distal part of the first catheter.
123. A kit for prevention of distal embolism during endovascular interventions, comprising:
   a first catheter, comprising:
      a proximal part and a
      distal part, having a
         principal through lumen,
         two lumens for filling two balloons,
         two balloons located at a distance from each other, one of which, the distal, is inflated and closes the lumen of the right or left common carotid artery, and the other, the proximal, is inflated and closes the lumen of the left or right subclavian artery, and
         anti-embolus filter provided at the proximal end of the principal through lumen of the distal part of the first catheter; and
   a second catheter, made in the form of a single-lumen tube, with a proximal end and distal end;
      in which the proximal and distal parts of the first catheter have external diameters of sufficient dimensions to allow passage through the lumen of the second catheter, and are of sufficient length to allow at least the distal part of the first catheter to extend beyond the limits of the distal end of the second catheter when the first catheter passes through the second catheter;
      in this case the internal diameter of the lumen of the second catheter exceeds the external diameter of the distal part of the first catheter;
      in this case, the part of the distal part of the catheter located between the balloons is made so as to allow blood to flow through said principal lumen but to block the passage of emboli through it.
124. A kit according to embodiment 123, in which
   the proximal part of the first catheter consists of two lumens for filling two balloons; and
   the distal part of the first catheter takes the form of a three-lumen tube, comprising:
      a principal through lumen of sufficient internal diameter to allow a flow of blood through the above-mentioned principal lumen, and
      two lumens for filling two balloons, which are narrower than the principal through lumen and connect with the previously mentioned two lumens for filling the two balloons in the proximal part of the first catheter.
125. A kit according to any one of embodiments 123-124, in which each lumen for filling of balloons in the distal part of the first catheter ends at the place of positioning of the corresponding balloon, and the principal through lumen passes through the place of positioning of the balloons as far as the distal end of the distal part of the first catheter.
126. A kit according to any one of embodiments 123-125, in which the balloons are made of an elastomer material.
127. A kit according to any one of embodiments 123-126, in which the distal part of the first catheter is made of radio-opaque material or contains at least one radio-opaque marker.
128. A kit according to any one of embodiments 123-127, in which the principal through lumen of the distal part of the first catheter has an internal diameter of not less than 1 mm but not more than 2.5 mm.
129. A kit according to any one of embodiments 123-128, in which the distal part of the first catheter has an external diameter of not less than 1.2 mm but not more than 2.8 mm.
130. A kit according to any one of embodiments 123-129, in which the proximal ends of the lumens for filling of balloons in the proximal part of the first catheter are closed with an elastic material through which an injection needle can pass.
131. A kit according to any one of embodiments 123-130, in which the length of the part of the distal part of the first catheter located between the balloons is 3-4 cm.
132. A kit according to any one of embodiments 123-131, in which the wall of the principal through lumen of the part of the distal part of the first catheter located between the balloons contains holes.
133. A kit according to embodiment 132, in which the above-mentioned holes made in the wall of the principal through lumen measure from 50 to 300 μm, and more preferably to 100 μm, in diameter.
134. A kit according to any one of embodiments 123-131, in which the part of the distal part of the first catheter located between the balloons takes the form of a braided, knitted or meshed polymer anti-embolus filter.
135. A kit according to embodiment 134, in which the holes in the above-mentioned anti-embolus filter, positioned in the part of the distal part of the first catheter located between the balloons, measure from 50 to 300 μm, and more preferably to 100 μm, in diameter.
136. A kit according to any one of embodiments 123-135, in which the anti-embolus filter, positioned at the proximal end of the principal through lumen of the distal part of the first catheter takes the form of a braided, knitted or meshed polymer filter the length of which ranges from 2 cm to 40 cm, preferably to 30 cm and more preferably to 20 cm.
137. A kit according to any one of embodiments 123-136, in which the holes in the anti-embolus filter positioned at the proximal end of the principal through lumen of the distal part of the first catheter measure 50 to 300 μm, more preferably to 100 μm.
138. A kit according to any one of embodiments 123-137, in which the anti-embolus filter positioned at the proximal end of the principal through lumen of the distal part of the first catheter contains an additional hole equivalent in diameter to that of the guidewire passing through said hole into the principal through lumen of the distal part of the first catheter.
139. A kit according to any one of embodiments 123-138, in which the distal end of the principal through lumen of the distal part of the first catheter is closed with an additional anti-embolus filter.
140. A kit according to any one of embodiments 123-139, additionally comprising:
   a third catheter in the form of a single-lumen tube with a proximal end and a distal end;
      in which case the proximal and distal parts of the first catheter have external diameters sufficient to allow passage through the lumen of the third category and lengths which together are sufficient to allow at least the distal part of the first catheter to extend beyond the limits of the distal end of the third catheter, when the first catheter passes through the third catheter;
      in which case the internal diameter of the lumen of the third catheter is essentially the same as the external diameter of the distal part of the first catheter.
141. A kit for prevention of distal embolism during endovascular interventions, comprising:
   a first catheter, comprising:
      a proximal part and a
      distal part, having a
         principal through lumen,
         two lumens for filling two balloons,
         two balloons placed at a distance from each other, one of which, the distal, is inflated and thus closes the lumen of the right or left common carotid artery, and the other, the proximal, is inflated and thus closes the lumen of the left or right subclavian artery; and
   a second catheter, made in the form of a single-lumen tube, with a proximal end and distal end;
      in which the proximal and distal parts of the first catheter have external diameters of sufficient dimensions to allow passage through the lumen of the second catheter, and are of sufficient length to allow at least the distal part of the first catheter to extend beyond the limits of the distal end of the second catheter when the first catheter passes through the second catheter;
      in this case the internal diameter of the lumen of the second catheter exceeds the external diameter of the distal part of the first catheter;
      in this case the part of the distal part of the first catheter positioned between the balloons is made to allow a flow of blood through the above-mentioned principal lumen but also to block the passage of emboli through it;
      in this case, the proximal end of the principal through lumen of the distal part of the first catheter is closed, with a hole equivalent in diameter to that of the guidewire passing through said hole into the principal through lumen of the distal part of the first catheter.
142. A kit according to embodiment 141, in which
   the proximal part of the first catheter consists of two lumens for filling two balloons; and
   the distal part of the first catheter takes the form of a three-lumen tube, comprising:
      a principal through lumen of sufficient internal diameter to allow a flow of blood through the above-mentioned principal lumen, and
      two lumens for filling two balloons, which are narrower than the principal through lumen and connect with the previously mentioned two lumens for filling the two balloons in the proximal part of the first catheter.
143. A kit according to either of embodiments 141 or 142, in which each lumen for filling the balloons in the distal part of the first catheter ends at the place of positioning of the corresponding balloon, and the principal through lumen passes through the place of positioning of the balloons as far as the distal end of the distal part of the first catheter.
144. A kit according to any one of embodiments 141-143, in which the balloons are made of elastomer material.
145. A kit according to any one of embodiments 141-144, in which the distal part of the first catheter is made of radio-opaque material or contains at least one radio-opaque marker.
146. A kit according to any one of embodiments 141-145, in which the principal through lumen of the distal part of the first catheter has an internal diameter of not less than 1 mm but not more than 2.5 mm.
147. A kit according to any one of embodiments 141-146, in which the distal part of the first catheter has an external diameter of not less than 1.2 mm but not more than 2.8 mm.
148. A kit according to any one of embodiments 141-147, in which the proximal parts of the lumens for filling the balloons in the proximal part of the first catheter are closed using an elastic material through which an injection needle can pass.
149. A kit according to any one of embodiments 141-148, in which the length of the part of the distal part of the first catheter located between the balloons is 3-4 cm.
150. A kit according to any one of embodiments 141-149, in which the wall of the principal through lumen of the part of the distal part of the first catheter located between the balloons contains holes.
151. A kit according to embodiment 150, in which the previously mentioned holes in the wall of the principal through lumen are 50 to 300 μm, and more preferably 100 μm, in diameter.
152. A kit according to any one of embodiments 141-149, in which the part of the distal part of the first catheter located between the balloons takes the form of a braided, knitted or meshed polymer anti-embolus filter.
153. A kit according to embodiment 152, in which the holes in the previously mentioned anti-embolus filter in the part of the distal part of the first catheter located between the balloons are 50 to 300 μm, and more preferably 100 μm, in diameter.
154. A kit according to any one of embodiments 141-153, in which the distal end of the principal through lumen of the distal part of the first catheter are closed with an additional anti-embolus filter.
155. Method of preventing distal embolism during endovascular interventions using a kit according to any one of embodiments 101-121, 123-139 and 141-154, including steps of:
   injecting the radial artery or brachial artery;
   inserting the introducer into the radial artery or brachial artery respectively;
   inserting the guidewire through the introducer into the left or right common carotid artery;
   inserting the first catheter via said guidewire through the previously mentioned introducer, so that the distal balloon is positioned in the left or right common carotid artery respectively, and the proximal balloon is positioned in the left or right subclavian artery;
   inflating, through one of the lumens used to fill the balloon, first the distal balloon of the above-mentioned first catheter until the distal balloon closes the lumen of the previously mentioned left or right common carotid artery respectively, and then inflating the proximal balloon of the above-mentioned first catheter through another lumen to fill the balloon until the proximal balloon closes the lumen of the previously mentioned left or right subclavian artery;
   placing, after the endovascular intervention is completed in the ascending thoracic aorta and the aortic arch, the second catheter on the first catheter;
   inflating the proximal balloon of the previously mentioned first catheter;
   bringing the previously mentioned second catheter to the inflated distal balloon in the previously mentioned first catheter and, by swift introduction of a liquid substance into the second catheter, flushing the emboli remaining in the area around the distal balloon in the first catheter into the distal blood flow in the left or right common carotid artery;
   removing the second catheter;
   inflating the distal balloon in the previously mentioned first catheter;
   removing the first catheter, guidewire and introducer, as mentioned above.
156. Method according to embodiment 155, where the liquid used for flushing the emboli is saline solution.
157. Method according to either of embodiments 155 or 156, in which the endovascular intervention is an endovascular aortic valve replacement.
158. Method of preventing distal embolism during endovascular interventions, using a kit according to either of embodiments 122 or 140, including steps of:
   injecting the radial artery or brachial artery;
   inserting the introducer into the radial artery or brachial artery respectively;
   inserting the guidewire through the introducer into the left or right common carotid artery;
   inserting the first catheter via the guidewire through the previously mentioned introducer so that the distal balloon is positioned in the left or right common carotid artery respectively, and the proximal balloon is positioned in the left or right subclavian artery;
   inflating, through one of the lumens used to fill the balloon, first the distal balloon of the above-mentioned first catheter until the distal balloon closes the lumen of the previously mentioned left or right common carotid artery respectively, and then inflating the proximal balloon of the above-mentioned first catheter through another lumen to fill the balloon until the proximal balloon closes the lumen of the previously mentioned left or right subclavian artery;
   inserting the third catheter via the introducer into the previously mentioned first catheter from the proximal end;
   inflating the proximal balloon of the previously mentioned first catheter;
   moving the previously mentioned third catheter along to the stop in the distal balloon of the first catheter;
   introducing the liquid medicinal preparation into the third catheter;
   removing the third catheter;
   inflating the proximal balloon of the previously mentioned first catheter until the lumen of the left or right subclavian artery is closed by the proximal balloon;
   placing, after the endovascular intervention is completed in the ascending thoracic aorta and the aortic arch, the second catheter on the first catheter;
   inflating the proximal balloon of the previously mentioned first catheter;
   bringing the previously mentioned second catheter to the inflated distal balloon in the previously mentioned first catheter and, by swift introduction of a liquid substance into the second catheter, flushing the emboli remaining in the area around the distal balloon in the first catheter into the distal blood flow in the left or right common carotid artery;
   removing the second catheter;
   inflating the distal balloon in the previously mentioned first catheter;
   removing the first catheter, guidewire and introducer, as mentioned above.
159. Method according to embodiment 158, where the liquid used to flush out the emboli is saline solution.
160. Method according to either of embodiments 158 or 159, in which the endovascular intervention is an endovascular aortic valve replacement.

What is claimed is:

1. A kit for prevention of distal embolism during endovascular interventions, comprising:
a first catheter, comprising:
a proximal part and a distal part,
at least one balloon, configured for closing a lumen of the right or left common carotid artery, and
a proximal end, configured to allow a flow of blood and to block a passage of emboli through a principal lumen at the distal part; and
a second catheter, having a form of a single-lumen tube and having a proximal end and a distal end;
wherein the proximal and distal parts of the first catheter have external diameters that allow passage through the single-lumen tube of the second catheter, and are of sufficient length to allow at least the distal part of the first catheter to extend beyond the distal end of the second catheter when the first catheter passes through the second catheter; and
wherein an internal diameter of the single-lumen tube of the second catheter exceeds an external diameter of the distal part of the first catheter.

2. The kit of claim 1, wherein
the proximal part of the first catheter is configured for filling at least a second balloon; and
the distal part of the first catheter has a form of a tube of sufficient internal diameter to allow said flow of blood through the principal lumen, and is configured for filling said at least one balloon, said at least one balloon being narrower than the principal lumen and connecting with at least one lumen for filling at least one balloon in the proximal part of the first catheter.

3. The kit of claim 1, wherein the distal part of the first catheter has at least one lumen for filling at least one balloon that ends at a location of positioning of the at least one balloon, and the principal lumen passes through the location of positioning as far as a distal end of the distal part of the first catheter.

4. The kit of claim 1, wherein the at least one balloon is made of an elastomer material.

5. The kit of claim 1, wherein the distal part of the first catheter comprises a radio-opaque material or a radio-opaque marker.

6. The kit of claim 1, wherein the at least one balloon is positioned on the side of a distal end of the distal part.

7. The kit of claim 6, wherein an anti-embolus filter is positioned on a proximal end of the principal through lumen.

8. The kit of claim 7, wherein the anti-embolus filter comprises a set of through holes made in the wall of the principal lumen at the proximal end of the principal through lumen.

9. The kit of claim 8, wherein the principal lumen of the distal part of the first catheter is cylindrical throughout its length.

10. The kit of claim 9, wherein the holes made in the wall of the principal through lumen are located at a distance of at least 20 cm and not more than 70 cm from the balloon and measure from 50 to 300 μm in diameter and more preferably up to 100 μm.

11. The kit of claim 8, wherein the principal through lumen of the distal part of the first catheter narrows conically at its proximal end.

12. The kit of claim 11, wherein a length of the conically narrowing proximal end of the principal through lumen of the distal part of the first catheter is within a range of 2 cm to 40 cm.

13. The kit of claim 11, wherein the holes made in the wall of the principal through lumen are 50 to 100 μm.

14. The kit of claim 7, wherein the anti-embolus filter is a braided, knitted, or meshed polymer conical filter.

15. The kit of claim 7, wherein the anti-embolus filter is a network that closes the proximal hole in the principal through lumen.

16. A method of preventing distal embolism during endovascular interventions using the kit of claim 1, comprising:
injecting the common femoral artery, the radial artery, or the brachial artery;
inserting an introducer into said common femoral artery, radial artery or brachial artery;
inserting a guidewire through the introducer into the left or right common carotid artery;
inserting the first catheter through the introducer using said guidewire;
inflating, through at least one lumen, the at least one balloon in said first catheter, until the lumen of said right or left common carotid artery is closed;
placing, after the endovascular intervention is completed in the ascending thoracic aorta and the aortic arch, the second catheter on the first catheter;
bringing said second catheter up to the inflated balloon in the first catheter and introducing a liquid substance into the second catheter to allow flushing of emboli remaining in areas around the balloon into the distal blood flow behind the aortic arch before the first catheter in the left or right common carotid artery;
removing the second catheter;
inflating at least one balloon in the first catheter;
removing the first catheter, guidewire and introducer, as mentioned above.

17. The method of claim 16, in which the kit including the first catheter, comprising one balloon and one lumen for filling the balloon, is used.

18. The method of claim 16, wherein the kit including the first catheter, comprising two balloons and two lumens for filling the balloons, is used.

19. The method of claim 16, where thein liquid used for flushing out the emboli is saline solution.

20. The method of claim 16, wherein the endovascular procedure is an endovascular aortic valve replacement.
